(19) United States
(12) Patent Application Publication
(10) Pub. No.: US 2026/0137294 A1
(43) Pub. Date: May 21, 2026

(54) BIOLOGICAL INFORMATION PROVIDING APPARATUS

(71) Applicant: Asahi Kasei Microdevices Corporation, Tokyo (JP)

(72) Inventors: Naoto TAMURA, Tokyo (JP); Shigeru IWASA, Tokyo (JP)

(21) Appl. No.: 19/331,973

(22) Filed: Sep. 17, 2025

(30) Foreign Application Priority Data
Sep. 18, 2024 (JP) 2024-161439

(51) Int. Cl. A61B 5/053; A61B 5/00; A61B 5/25; A61B 5/30

(52) U.S. Cl. CPC A61B 5/053; A61B 5/25; A61B 5/30; A61B 5/721; A61B 5/725; A61B 2562/0219

(57) ABSTRACT

Provided is a biological information providing apparatus comprising a potential signal measuring unit which measures a potential signal through a pair of electrodes in contact with a living body, a bioimpedance signal measuring unit which outputs a bioimpedance signal according to the bioimpedance, an adaptive filter having a variable coefficient filtering unit which generates a control signal, a biological information signal output unit which calculates a biological information signal, and a coefficient adjusting unit which adjusts the coefficient such that a signal correlated to the bioimpedance signal included in the biological information signal is reduced, and a filter control unit which controls the coefficient adjusting unit such that a convergence speed of the adaptive filter becomes faster when the potential signal or the bioimpedance signal satisfies a predetermined condition.

### BACKGROUND

### 1. Technical Field

The present invention relates to a biological information providing apparatus.

### 2. Related Art

Patent document 1 describes an electrode system for reducing a motion artifact from a potential signal. Patent document 2 describes an active type noise reduction device which reduces residual abnormal sound when applying an active type noise reduction device to an in-cabin environment where audio signals are reproduced. Patent document 3 describes a demodulator having a zone detection section that detects a replacement target zone to be replaced and a replacement section that replaces a signal of the replacement target zone with a replacement target signal.

### RELATED ART DOCUMENTS

### Patent Documents

Patent Document 1: U.S. Patent Application Publication No. 20030171661
Patent Document 2: Japanese Patent Application Publication No. 2018-154173
Patent Document 3: Japanese Patent No. 6413023

### BRIEF DESCRIPTION OF THE DRAWINGS

FIG. 1 is an example of a schematic diagram illustrating a bioimpedance BioZ generated in a living body 500.

FIG. 2 illustrates an example of a block diagram of a configuration of a biological information providing apparatus 100 according to a comparative example.

FIG. 3 illustrates an example of a block diagram of an equivalent circuit showing an algorithm of the configuration of the biological information providing apparatus 100 according to the comparative example.

FIG. 4A illustrates a simulation result of a potential signal d (n) and a biological information signal e (n) after the noise is removed, when μ=0.2 is set.

FIG. 4B illustrates a simulation result of the potential signal d (n) and the biological information signal e (n) after the noise is removed, when μ=1.0 is set.

FIG. 5 illustrates an example of a block diagram of a configuration of a biological information providing apparatus 200a according to a first embodiment.

FIG. 6 is an example of a flow diagram showing the processing of the biological information providing apparatus 200a according to the first embodiment.

FIG. 7 illustrates an example of a graph in a case where switching among a plurality of step-size parameters p is performed according to a plurality of thresholds.

FIG. 8 illustrates an example of a block diagram of a configuration of a biological information providing apparatus 200b according to a second embodiment.

FIG. 9A is an example of a flow diagram showing the processing of the biological information providing apparatus 200b according to the second embodiment.

FIG. 9B is an example of a flow diagram further detailing step S204 of FIG. 9A.

FIG. 10 illustrates an example of a block diagram of a configuration of a biological information providing apparatus 200c according to a third embodiment.

FIG. 11 is an example of a flow diagram showing the processing of the biological information providing apparatus 200c according to the third embodiment.

FIG. 12 illustrates an example of a block diagram of a configuration of a biological information providing apparatus 200d according to a fourth embodiment.

FIG. 13A is an example of a graph for describing the functionality of a filter control unit 60d.

FIG. 13B is an example of a graph for describing the functionality of the filter control unit 60d.

FIG. 13C is an example of a graph for describing the functionality of the filter control unit 60d.

FIG. 13D is an example of a graph for describing the functionality of the filter control unit 60d.

FIG. 13E is an example of a graph for describing the functionality of the filter control unit 60d.

FIG. 13F is an example of a graph for describing the functionality of the filter control unit 60d.

FIG. 14 is an example of a flow diagram showing the processing of the biological information providing apparatus 200d according to the fourth embodiment.

FIG. 15 illustrates an example of a block diagram of a configuration of a biological information providing apparatus 200e according to a fifth embodiment.

FIG. 16 is an example of a flow diagram showing processing of the biological information providing apparatus 200e according to the fifth embodiment.

FIG. 17 illustrates an example of a block diagram of a configuration of a biological information providing apparatus 200f according to a sixth embodiment.

FIG. 18 illustrates an example of a block diagram of a configuration of a biological information providing apparatus 200g according to a seventh embodiment.

FIG. 19 is a flow diagram showing an example of processing for sudden noise removal performed at the biological information providing apparatus 200g.

FIG. 20 illustrates an example of a block diagram of a configuration of a biological information providing apparatus 200h according to an eighth embodiment.

FIG. 21 illustrates an example of a block diagram of a configuration of a biological information providing apparatus 450 according to a comparative example.

FIG. 22 illustrates an example of a block diagram of a configuration of a biological information providing apparatus 400 according to a ninth embodiment.

FIG. 23 illustrates an example of a block diagram of a configuration of a biological information providing apparatus 400 according to a tenth embodiment.

FIG. 24 illustrates an example of a block diagram of a configuration of a biological information providing apparatus 400 according to an eleventh embodiment.

FIG. 25 is an example of a flow diagram showing the processing of the biological information providing apparatus 400 according to the ninth embodiment.

FIG. 26 illustrates an example of a block diagram of a configuration of a biological information providing apparatus 400 according to a twelfth embodiment.

FIG. 27 illustrates an example of a block diagram of a configuration of a biological information providing apparatus 400 according to a thirteenth embodiment.

FIG. 28 is an example of a flow diagram showing processing of the biological information providing apparatus 400 according to the twelfth embodiment.

### DESCRIPTION OF EXEMPLARY EMBODIMENTS

The present invention will be described below through embodiments of the invention, but the following embodiments do not limit the invention according to the claims. In addition, not all combinations of features described in the embodiments are essential to a solution of the invention. Hereinafter, although each of the embodiments may be distinguished by providing the embodiment with an ordinal number in order to describe the embodiment, the ordinal number is provided only for convenience of description, and it is not to eliminate a combination of configurations of the embodiment shown as having different ordinal numbers. For example, a part of the configuration of the first embodiment may be used by adding, replacing, and/or combining a part of the configuration of the third embodiment, as needed.

In the present specification, the term “connect” may be used to refer to an electrically connected relationship between components. In this case, the term “connect” may refer to direct connection to a component of interest through conducting wire, but it is not intended to eliminate indirect connection with another component in between thereof.

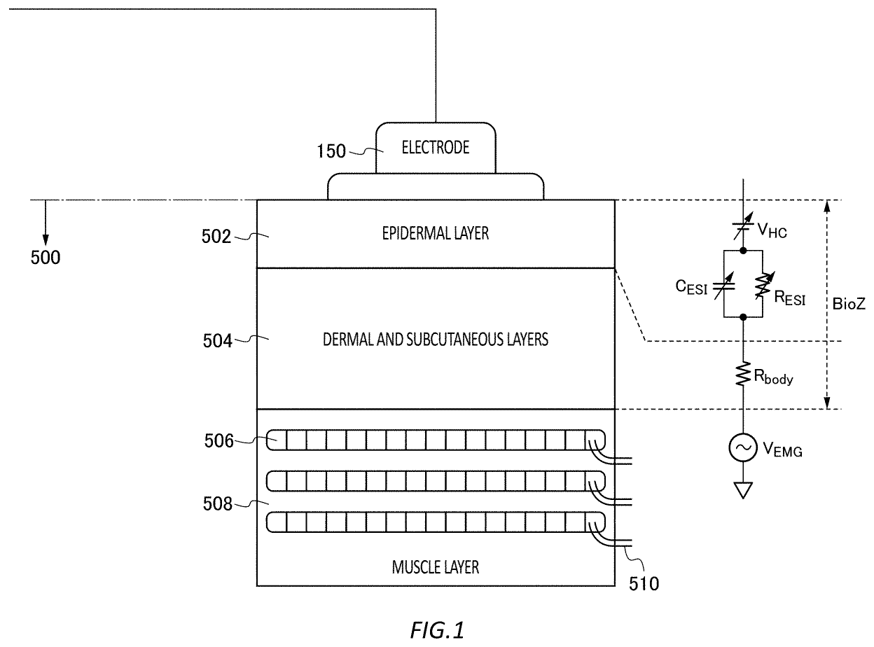

FIG. 1 is an example of a schematic diagram illustrating a bioimpedance BioZ generated in a living body 500. The living body 500 includes an epidermal layer 502, dermal and subcutaneous layers 504, and a muscle layer 506.

The living body 500 of the present embodiment is a human. However, the living body 500 may be another animal. In a case where the living body 500 is another animal, some skin structures may differ depending on the type of the living body 500.

In the measurement of the bioimpedance BioZ, a pair of electrodes 150 are brought into contact with and fixed to the living body 500, and an AC current is passed between the electrodes. In this case, the bioimpedance BioZ based on the body composition of the living body 500 can be read by reading a potential difference generated between the pair of electrodes 150. In this manner, the AC signal applied to the living body 500 via the pair of electrodes 150 may be a differential signal based on a voltage and current or the like applied between the electrodes.

Note that, the AC signal applied to the living body 500 may be a signal obtained by periodically varying a level of voltage to be applied to one electrode of the pair of electrodes 150, relative to a ground voltage. In this case, the bioimpedance BioZ can be read via a measurement apparatus connected to another electrode of the pair of electrodes 150.

In addition, for example, in a case where the living body 500 activates muscles, an action potential is generated by electrical excitation of cells in the muscle layer 506 of the living body 500. In the measurement of such an action potential, since a variation in a contact impedance has a strong influence thereon, an impedance caused by the epidermal layer 502 and the dermal and subcutaneous layers 504 will have a strong influence.

The epidermal layer 502 is an epidermis of the living body 500. For example, in a case where the living body 500 is a human, the epidermal layer 502 is a portion having an average thickness of about 0.2 mm in the skin of a portion other than a palm or the sole of a foot.

The epidermal layer 502 contributes as a half-cell potential V_{HC} and a variable resistance R_{ESI} and a variable capacitance C_{ESI} connected in series to the half-cell potential V_{HC} in the measurement of the bioimpedance BioZ by the electrode 150. The half-cell potential V_{HC} is an electrostatic potential generated at a portion where the electrode 150 and the epidermal layer 502 are in contact with each other. The component of the half-cell potential V_{HC} contributes, for example, as a component having a frequency of 20 Hz or less in the measurement of the bioimpedance BioZ.

Herein, in the bioimpedance BioZ, the contribution of a contact impedance between the electrode 150 and the skin of the living body 500, that is, between the electrode 150 and the epidermal layer 502 is large. Furthermore, the epidermal layer 502 contributes as the variable resistance R_{ESI} and the variable capacitance C_{ESI} connected in series to the half-cell potential V_{HC}. The variable resistance R_{ESI} and the variable capacitance C_{ESI} vary significantly based on changes in the state of the skin surface and a contact state between the electrode 150 and the skin. This appears as a variation in the contact impedance between the electrode 150 and the skin of the living body 500 in the bioimpedance BioZ. In a state where the skin surface is dry, these impedances increase by about 10 times, and thus are expressed as variable resistances and capacitances in an equivalent circuit.

The dermal and subcutaneous layers 504 are layers such as a dermal layer, a subcutaneous tissue, and a fascia. In the measurement of the bioimpedance, the dermal and subcutaneous layers 504 contribute as a resistance R_{body}. Of the dermal and subcutaneous layers 504, the dermal layer is a site of the skin through which capillaries, lymphatic vessels, nerves, and the like pass, and is a portion formed inside the epidermal layer 502. For example, in a case where the living body 500 is a human, the dermal layer is a portion having an average thickness of about 2 mm. Of the dermal and subcutaneous layers 504, the subcutaneous tissue is formed further inside the dermal layer. The subcutaneous tissue is a portion that supports the epidermal layer 502 and the dermal layer, and is a portion having an average thickness of about 2 mm to about 9 mm. The subcutaneous layer mainly contains fat cells and includes large blood vessels and the like. Furthermore, the dermal and subcutaneous layers 504 include the fascia between the subcutaneous tissue and the muscle layer 506. The fascia has a thickness of about 1 mm and is a portion that generates an electrical resistance, although not as large as the skin. The resistance R_{body} of the dermal and subcutaneous layers 504 is a resistance value obtained by adding the electrical resistances of the plurality of layers.

As a result, in the impedance measurement of the living body 500, what contributes as the bioimpedance BioZ is the variable resistance R_{ESI} and the variable capacitance C_{ESI} connected in parallel, and the resistance R_{body}.

The muscle layer 506 is a layer including a plurality of muscle fibers 508. The muscle fibers 508 are tissues that are activated by electrical signals transmitted through nerves 510. When the muscle fibers 508 move, action potentials are generated in the muscle fibers 508. In the muscle layer 506, a potential V_{EMG} (composite action potential V_{EMG}) obtained by adding (combining) action potentials of a plurality of muscle fibers is generated according to the activities of a plurality of muscles. In the measurement of an electromyogram signal (EMG), such a composite action potential V_{EMG} is measured.

Since such action potential V_{EMG} is a potential generated through the activity of the living body, the action potential V_{EMG} is a potential difference that can be generated between a pair of electrodes 150 attached to the living body 500 even in a case where the energization from the outside is not performed. However, the electromyogram signal is a signal having a low potential, and thus in a case where the bioimpedance BioZ is measured together when such a electromyogram signal is measured, a signal which applies a potential difference between the pair of electrodes 150 is output from the outside of the living body 500.

The electromyogram signal is an example of a “biological information signal” indicating information related to the living body. The electromyogram signal may have a peak of amplitude in a frequency band higher than 20 Hz and 4 kHz or less, for example. As another example, the biological information signal may be a signal indicating a potential generated through the activity of the living body, such as electrocardiogram (ECG), electroencephalogram (EEG), or electro-oculogram (EOG). Note that these biological information signals are examples, and the biological information signal is not limited to these signals as long as it is a signal based on the action potential of the living body 500 generated through the activity of the living body 500.

Herein, in the measurement of the action potential V_{EMG}, in a case where the living body 500 moves, the electrode 150 is displaced with respect to the living body 500, or the living body 500 vibrates, so that a motion artifact (MA) generated at a contact point between the electrode 150 and the living body 500 may occur as measurement noise. In the measurement of the bioimpedance BioZ, it is known that a component proportional to the MA is included in a variation component of the bioimpedance BioZ. Therefore, when measuring the action potential V_{EMG}, measurement of the action potential V_{EMG} that is not affected by the measurement noise of the motion artifact can be performed by subtracting the variation component of the bioimpedance BioZ proportional to the motion artifact. In addition, for example, in the measurement of the action potential V_{EMG}, a three-dimensional location of where the action potential V_{EMG} is generated can also be identified by using a plurality of measurement apparatuses, when the plurality of measurement apparatuses are arranged with predetermined intervals in a circumferential direction of the arm. The relationship between the electromyogram signal and a gesture of the arm can thereby be measured, and there is a need for accurately performing the measurement of the electromyogram signal.

The MA generated in a case where the living body 500 moves often has a peak at less than 20 Hz, for example, in a case where the living body 500 is a human. The influence of such an MA can be shielded by a high-pass filter. However, even when the frequency band in which the peak of the MA appears is a frequency band of 20 Hz or less, in a case where the peak value of the MA is large, the influence of the MA extending from the peak may appear in the frequency band of 20 Hz or more.

On the other hand, the vibration generated in the living body 500 is, for example, vibration or the like generated in a moving body, such as a train, a bus, or an airplane, on which a human is boarding. The MA generated when the living body 500 boards the moving body can be mixed directly as noise in a frequency band of 50 Hz or more and less than 200 Hz, for example. In this case, the MA is known to contribute as a variation of a capacitive component, and contributes as a component proportional to the variation component of the C_{ESI}. Furthermore, the variation in the bioimpedance BioZ caused by the MA may have a frequency greater than a predetermined frequency (for example, 20 Hz).

Therefore, in the measurement of the variable capacitance C_{ESI}, by shielding components in an appropriate frequency range and amplifying the shielded components, the variation in a capacitive element proportional to the MA can be read, and thus, the variation in the bioimpedance BioZ caused by the MA can be read. Hereinafter, some configurations of a biological information providing apparatus capable of reading such variations in the variable capacitance C_{ESI} proportional to the MA will be described in detail. In particular, effect of the biological information providing apparatus performing noise removal when a noise to the potential signal due to the MA is suddenly generated will be described in detail.

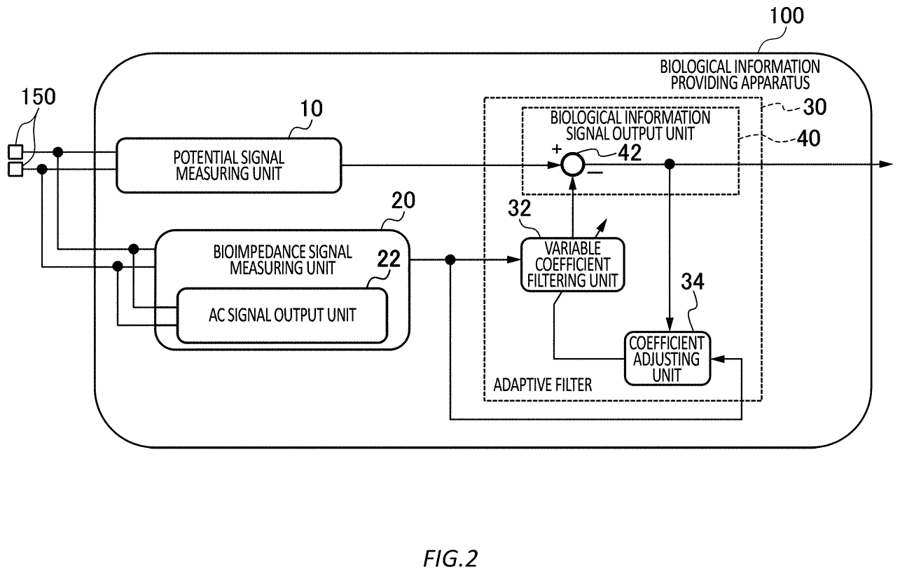

FIG. 2 illustrates an example of a block diagram of a configuration of a biological information providing apparatus 100 according to a comparative example. The biological information providing apparatus 100 is configured by including a potential signal measuring unit 10, a bioimpedance signal measuring unit 20, and an adaptive filter 30.

The potential signal measuring unit 10 measures the potential signal through a pair of electrodes 150 in contact with a living body 500. The potential signal measuring unit 10 may amplify a composite action potential measured through the pair of electrodes 150 and output, as a potential signal, a signal obtained by performing analog-to-digital conversion thereon.

The bioimpedance signal measuring unit 20 measures the bioimpedance generated between the pair of electrodes 150 to output a bioimpedance signal according to the bioimpedance BioZ. The bioimpedance signal measuring unit 20 outputs the bioimpedance signal to the adaptive filter 30. The bioimpedance signal measuring unit 20 includes an AC signal output unit 22.

The AC signal output unit 22 applies an AC signal to the pair of electrodes 150 in order for the bioimpedance signal measuring unit 20 to measure the bioimpedance BioZ. For example, the AC signal output unit 22 includes a DC constant voltage source or a constant current source, and a mixer for generating an AC constant voltage signal. As another example, an AC power source with a square wave or sine wave and a resistor for limiting the amplitude of a signal are included.

The AC signal output unit 22 may apply an AC signal as a differential signal to the pair of electrodes 150. In this case, the bioimpedance signal measuring unit 20 measures the bioimpedance BioZ by detecting an impedance with respect to the differential signal. In another example, the AC signal output unit 22 applies, to one of the pair of electrodes 150, a waveform signal with a sine wave or square wave-shaped potential difference from a predetermined reference potential. In this case, each of the potential signal measuring unit 10 and the bioimpedance signal measuring unit 20 may include an amplifier and a single-ended to differential conversion circuit, and measure the bioimpedance BioZ by extracting a differential signal from a single-ended signal.

The adaptive filter 30 is a filter that removes, from the potential signal V_{EMG} generated by the composite action potential measured by the potential signal measuring unit 10, noise caused by the variation in the bioimpedance signal. The adaptive filter 30 is configured to include a variable coefficient filtering unit 32, a coefficient adjusting unit 34, and a biological information signal output unit 40.

The variable coefficient filtering unit 32 generates a control signal for removing the noise from the potential signal based on the bioimpedance signal and a coefficient. The variable coefficient filtering unit 32 may generate the control signal by multiplying the bioimpedance signal by a coefficient generated by the coefficient adjusting unit 34.

The coefficient adjusting unit 34 adjusts the coefficient of the variable coefficient filtering unit 32 based on the bioimpedance signal and the signal output by the biological information signal output unit 40. According to feedback based on the output of the biological information signal output unit 40, the coefficient adjusting unit 34 performs an adjustment of the coefficient adaptively relative to the noise that is mixed in the potential signal.

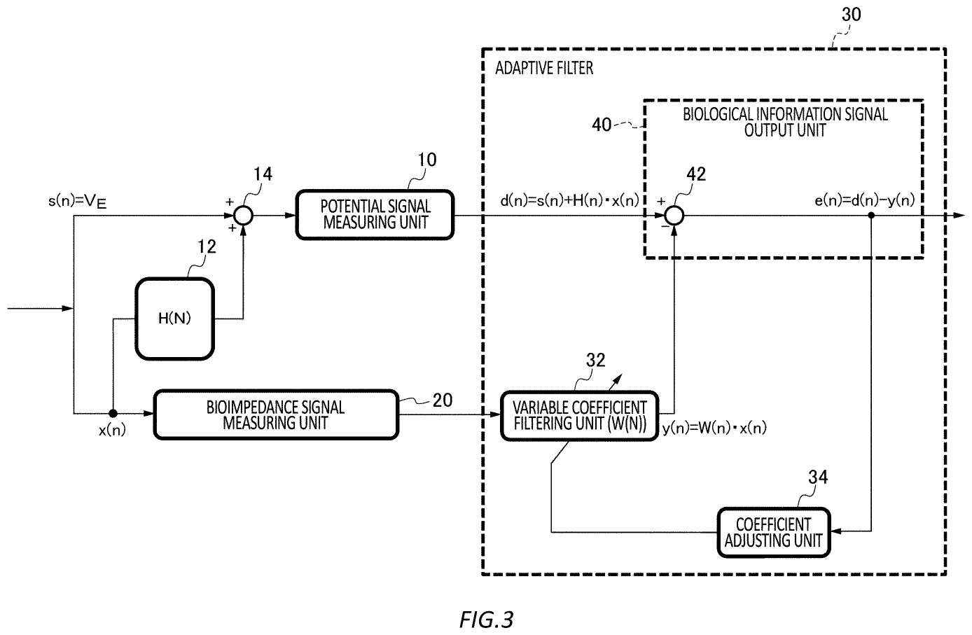

The biological information signal output unit 40 calculates a biological information signal obtained by removing a component of the control signal from the potential signal based on the potential signal output from the potential signal measuring unit 10 and the control signal output by the variable coefficient filtering unit 32, and outputs the calculated biological information signal. The biological information signal output unit 40 includes a subtractor 42, and has a path for outputting an output from the subtractor 42 to the outside and a path for performing feedback to adjust the variable coefficient filtering unit 32. Specific algorithms of the variable coefficient filtering unit 32 and the coefficient adjusting unit 34 will be described in detail with reference to FIG. 3.

FIG. 3 illustrates an example of a block diagram of an equivalent circuit showing an algorithm of the configuration of the biological information providing apparatus 100 according to the comparative example.

The potential signal measuring unit 10 acquires a biopotential signal from the electrode 150. In this case, as the signal from the electrode 150, signals on which the noise due to the MA correlated to the bioimpedance signal is superimposed are summed, and the potential signal measuring unit 10 outputs the summed signals as the potential signal d(n). Herein, in the measurement of the action potential V_{EMG}, a signal indicating only the “true” action potential to which any noise due to the bioimpedance BioZ is not mixed is determined to be V_{E}. A signal s(n), which is one of the input signals to be summed and input to the potential signal measuring unit 10, satisfies s(n)=V_{E}. Noise x(n) due to the bioimpedance BioZ, which is an input signal to be summed and input other than the signal s(s) input to the potential signal measuring unit 10, is mixed via an “unknown” coefficient H(n) shown in a noise mixture portion 12. Herein, n is a natural number representing a number of loop cycles of a feedback loop described below, which is a variable according to time.

Therefore, the potential signal d(n) output by the potential signal measuring unit 10 can be considered to be a signal obtained by adding H(n)×x(n) to s(n) via a parasitic adder 14. That is, the potential signal measuring unit 10 outputs the potential signal d(n) that satisfies d(n)=s(n)+N(n)×x(n).

On the other hand, the bioimpedance signal measuring unit 20 is connected to the electrode 150 similarly to the potential signal measuring unit 10, but can extract a signal x(n) based on the BioZ by filtering a component of another signal. The bioimpedance signal measuring unit 20 outputs the extracted signal x(n) to the variable coefficient filtering unit 32 and the coefficient adjusting unit 34.

The variable coefficient filtering unit 32 outputs a control signal W(n)×x(n) obtained by multiplying x(n) by a coefficient W(n). The coefficient W(n) is a coefficient having a value adjusted by the feedback loop via the coefficient adjusting unit 34 as the coefficient with a magnitude according to the output by the biological information providing apparatus 100.

The biological information signal output unit 40 outputs, based on the potential signal d(n) and the control signal W(n)×w(n), a biological information signal e(n), which is an external output from the biological information providing apparatus 100, and also outputs said signal to the coefficient adjusting unit 34 as a feedback for adjusting the coefficient of the variable coefficient filtering unit 32. The biological information signal output unit 40 includes the subtractor 42.

The subtractor 42 outputs the biological information signal e(n) obtained by subtracting the control signal W(n)×x(n) from the potential signal d. Therefore, the subtractor 42 outputs the signal e(n) that satisfies e(n)=d(n)−W(n)×x(n)=s(n)+H(n)×x(n)−W(n)×x(n). The biological information signal output unit 40 outputs the signal e(n) to the outside of the biological information providing apparatus 100 and to the coefficient adjusting unit 34.

The coefficient adjusting unit 34 adjusts the coefficient W(n) of the variable coefficient filtering unit 32 to adjust the magnitude of the coefficient of the adaptive filter 30. Then, the coefficient adjusting unit 34 adjusts the coefficient W(n) in order to adjust the magnitude of the control signal W(n)×x(n), such that a signal (H(n)×x(n)−W(n)×x(n)) correlated to a bioimpedance signal x(n) included in the biological information signal e(n)=s(n)+H(n)×x(n)−W(n)×x(n) is reduced. The coefficient adjusting unit 34 may adjust the coefficient W(n) in order to adjust the magnitude of the control signal W(n)×x(n), such that an error signal (H(n)×x(n)−W(n)×x(n)) correlated to a bioimpedance signal x(n) included in the biological information signal e(n)=s(n)+H(n)×x(n)−W(n)×x(n) becomes zero.

Herein, the signal can be expressed by elements such as an amplitude, a frequency, and a waveform. In the present specification, the “magnitude of a signal” for each signal may mean the magnitude of the amplitude of each signal, as long as it is not specifically mentioned otherwise. In particular, in a case of an AC signal, which is a differential signal, the signal has a positive or negative value, but the “magnitude of the signal” may mean the magnitude of the amplitude of the signal indicated by an absolute value.

In this manner, based on the feedback of the output signal, the variable coefficient filtering unit 32 and the coefficient adjusting unit 34 implements an adaptation algorithm in which the noise component is removed as the number of steps of the loop of the feedback loop is increased to adapt the output signal to be s(n). That is, the coefficient adjusting unit 34 performs the feedback such that W(n)=H(n) is established and the signal e(n) is converged to approach s(n) asymptotically, when the number of loop cycles n of the feedback loop is increased.

In this manner, the variable coefficient filtering unit 32 generates the control signal W(n)×x(n) obtained by performing adaptation according to the current error from the signal s(n) included in the signal e(n), such that the signal e(n) asymptotically approaches the signal s(n) indicating only the “true” action potential. In this regard, the adaptive filter 30 is referred to as the “adaptive” filter. As a result, the variable coefficient filtering unit 32 generates the control signal W(n)×x(n) indicating the noise component H(n)×x(n) included in the potential signal d, based on the bioimpedance signal x(n) and the coefficient W(n).

Herein, as the adaptation algorithm for the coefficient adjusting unit 34 to adjust the coefficient W, the approach of least mean square (LMS) algorithm may be used. In this case, the coefficient adjusting unit 34 is set, by using a parameter μ, which is called a step-size parameter, such that a recurrence formula W(n+1)=W(n)+μ×e(n)×x(n) is established for the coefficient W.

Likewise, an approach of normalized least mean square (NLMS) algorithm may be used as the adaptation algorithm. The normalized least mean square sets the coefficient W(n) such that the recurrence formula (1) is established.

$$W ( n + 1 ) = W ( n ) + μ \cdot e ( n ) \frac{x ( n )}{ε + ‖ x ( n ) ‖} \qquad ( 1 )$$

Herein, the constant ε is a constant known as a stability constant used to stabilize the numerical calculation. The NLMS approach is faster in the convergence speed of establishing W(n)=H(n) compared to the LMS approach, when using the step-size parameter μ, which is a constant.

As an example, the step-size parameter μ is a parameter having a value within a range of 0<μ<2.0. In the biological information providing apparatus 100 according to a comparative example, the step-size parameter μ is a constant with a value in this range.

The step-size parameter μ is an update amount of the coefficient of the variable coefficient filtering unit 32, that is, a parameter that controls a convergence speed of the adaptive filter 30. If the step-size parameter μ is too large, not only the control signal W(n)×x(n) but the magnitude of a signal output as a main signal e(n) will be attenuated. This is because if the step-size parameter μ is too large, the coefficient of the variable coefficient filtering unit 32 changes faster than the main signal e(n), which causes difficulty in distinguishing the main signal e(n) and the control signal W(n)×x(n) in the feedback via the coefficient adjusting unit 34, and causes a coefficient that attenuates the main signal together to be output in the adjustment of W(n) in the next step.

On the other hand, the convergence accuracy of the adaptive filter 30 is improved if the step-size parameter μ is small, but the convergence speed of the adaptive filter 30 becomes slow. However, when sudden noise is mixed in the bioimpedance signal x(n) in a case where the step-size parameter μ is small, the adaptive filter 30 may not be able to follow it at a sufficient speed.

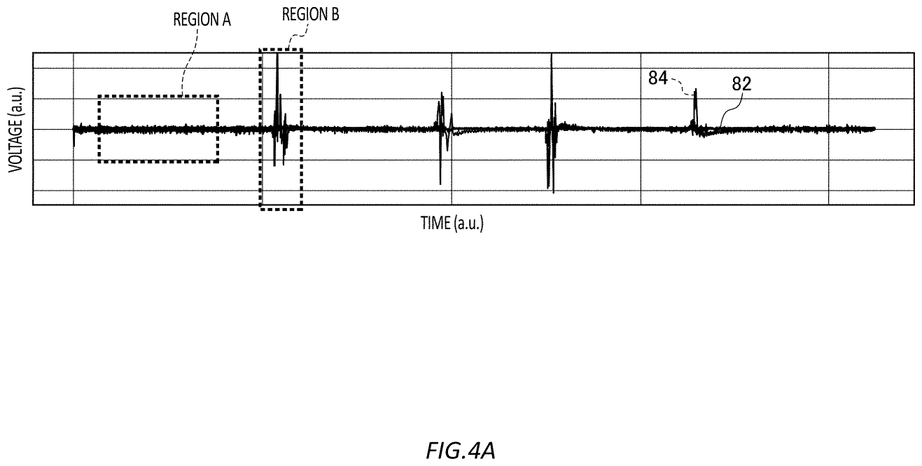

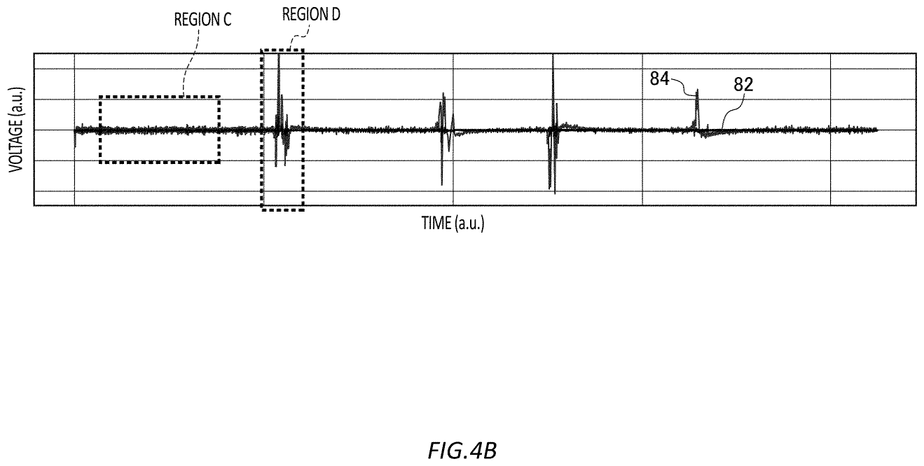

FIG. 4A illustrates a simulation result of a potential signal d (n) and a biological information signal e (n) after the noise is removed, when μ=0.2 is set. In the figure, a polygonal line 82 indicating the biological information signal e(n) after the noise is removed from the potential signal d(n) by the adaptive filter 30 and a polygonal line 84 indicating the potential signal d(n), and a region A and a region B are illustrated. Herein, the graph is intended to describe a summary and nature, and in the graph, the unit of each axis is shown by an arbitrary unit.

In the region A, the difference between the polygonal line 82 indicating the biological information signal e(n) after the noise is removed by the adaptive filter 30 and the polygonal line 84 indicating the potential signal d(n) is not large. In the figure, a signal of a desired magnitude is also obtained in the polygonal line 82 indicating the biological information signal e(n).

In the region B, large noise is included in the polygonal line 84 indicating the potential signal d(n). Therefore, the influence of the noise is not sufficiently removed in the polygonal line 82 indicating the biological information signal e(n) after the noise is removed by the adaptive filter 30. In this manner, in the region A in the figure, the step-size parameter μ is set to have a appropriate magnitude since the potential signal d(n) can be obtained with an appropriate magnitude. However, in the region B, since the step-size parameter μ is too small, the influence of the noise is not sufficiently removed.

FIG. 4B illustrates a simulation result of the potential signal d (n) and the biological information signal e (n) after the noise is removed, when μ=1.0 is set. In the figure, similar to FIG. 4A, a polygonal line 84 indicating the potential signal d(n) and a polygonal line 82 indicating the biological information signal e(n) after the noise is removed by the adaptive filter 30, and a region C and a region D are illustrated.

In region C, in contrast to the polygonal line 84 indicating the potential signal d(n), the polygonal line 82 indicating the biological information signal e(n) after the noise is removed by the adaptive filter 30 is attenuated greater than the region A in FIG. 4A. In the polygonal line 82 indicating the biological information signal e(n) after the noise is removed by the adaptive filter 30, the magnitude of the biological information signal e(n), which is the main signal, itself is also attenuated, which is an undesired operation.

In the region D, in the polygonal line 84 indicating the biological information signal e(n) after the noise is removed by the adaptive filter 30, the influence of the noise is sufficiently removed. In this manner, in the region C in the figure, the magnitude of the biological information signal e(n), which is the main signal, itself is also attenuated, and the step-size parameter μ is set to a value that is too large. On the other hand, in the region D, the influence of the noise is sufficiently removed, and the step-size parameter μ is set to an appropriate value.

As motion artifacts, there are steady ones and sudden ones. When the step-size parameter μ is set to a small value to handle a steady motion artifact, the noise may not be sufficiently removed when a sudden motion artifact is mixed, such as in the example shown in the region B in FIG. 4A. On the other hand, when the step-size parameter μ is set to a large value to handle a sudden motion artifact being mixed, the magnitude of the biological information signal e(n), which is the main signal, itself may be attenuated, such as in the example shown in the region C in FIG. 4B.

As described above, when the step-size parameter μ is set at a constant, an appropriate value of the step-size parameter μ is different for a region where sudden noise exists and a region where it does not exist. Therefore, when the step-size parameter μ is set at a constant, the step-size parameter μ may not be set at an appropriate value for each time region.

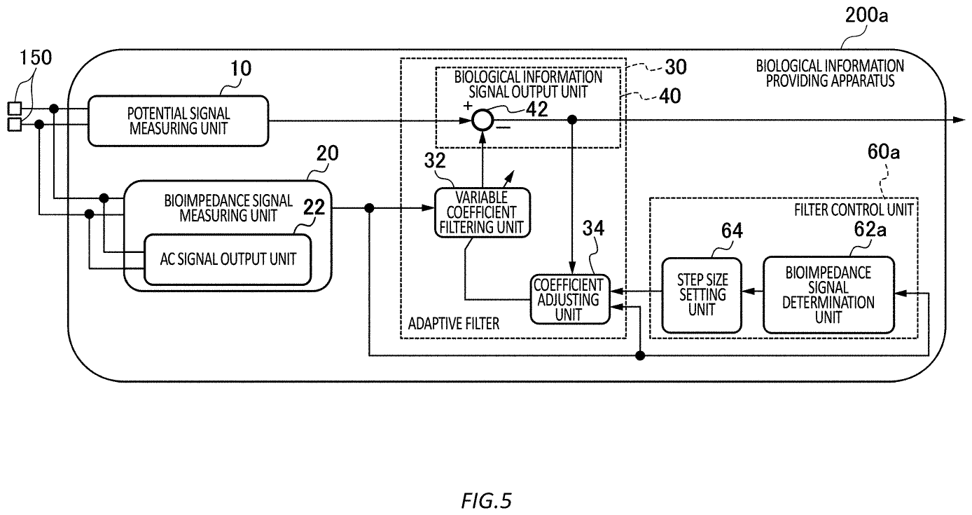

FIG. 5 illustrates an example of a block diagram of a configuration of a biological information providing apparatus 200a according to a first embodiment. Hereinafter, the configuration of the biological information providing apparatus 200a will be described focusing on a difference thereof from the biological information providing apparatus 100. The biological information providing apparatus 200a includes a filter control unit 60a in addition to the components included in the biological information providing apparatus 100.

The filter control unit 60a controls the adaptive filter 30 such that the convergence speed of the adaptive filter 30 is faster in a case where the bioimpedance signal x(n) satisfies a predetermined condition than in a case where the bioimpedance signal x(n) does not satisfy the condition. The filter control unit 60a includes a bioimpedance signal determination unit 62a and a step size setting unit 64.

The bioimpedance signal determination unit 62a determines whether or not the magnitude of the bioimpedance signal x(n) satisfies the predetermined condition. Herein, for each signal, “satisfying the predetermined condition” may mean that the amplitude expressed by an absolute value of each signal, as an example, is greater than a predetermined threshold. In this case, defining this threshold as a first threshold value, the “predetermined condition is satisfied” when the amplitude of the signal of interest is outside a range of [−first threshold value, +first threshold value]. In this example, the filter control unit 60a may determine, through determination by the bioimpedance signal determination unit 62a, that the bioimpedance signal x(n) satisfies the predetermined condition when the magnitude of the bioimpedance signal x(n) exceeds the predetermined first threshold value. In response to this determination, the convergence speed of the adaptive filter 30 is adjusted to reduce the control signal W(n)×x(n) according to the noise component that appears in the biological information signal e(n).

The step size setting unit 64 sets the magnitude of the step-size parameter μ based on the determination result of the bioimpedance signal determination unit 62a. Specifically, in the example of the determination described for the bioimpedance signal determination unit 62a, the step size setting unit 64 controls the adaptive filter 30 such that the step-size parameter μ of the adaptive filter 30 is set to a predetermined first value at the coefficient adjusting unit 34, when the magnitude of the bioimpedance signal x(n) exceeds the predetermined first threshold value. As an example, the first value may be μ=1.0, or may be μ=0.8. On the other hand, the step size setting unit 64 controls the adaptive filter 30 such that the step-size parameter μ of the adaptive filter 30 is set at a predetermined second value that is smaller than the first value at the coefficient adjusting unit 34, when the magnitude of the bioimpedance signal x(n) is equal to or less than the predetermined first threshold value. As an example, the second value may be μ=0.1, or may be μ =0.2. In this manner, in the present embodiment, instead of using a constant step-size parameter μ, different step-size parameters μ are used according to the condition.

In the present embodiment, through the adjustment of the coefficient by the coefficient adjusting unit 34, the step size setting unit 64 performs adjustment of the step-size parameter μ, when adjusting the coefficient. Note that, the step size setting unit 64 may perform the adjustment by directly controlling the variable coefficient filtering unit 32.

The filter control unit 60a thereby controls the coefficient adjusting unit 34 such that the step-size parameter μ of the coefficient adjusting unit 34 is set to the predetermined first value, when the bioimpedance signal x(n) satisfies the predetermined condition. The filter control unit 60a thereby adjusts the convergence speed of the adaptive filter 30 such that the influence of the control signal W(n)×x(n) according to the noise component that appears in the biological information signal e(n) is reduced. On the other hand, the filter control unit 60a may control the coefficient adjusting unit 34 such that the step-size parameter μ of the adaptive filter 30 is set at the predetermined second value that is smaller than the first value, when the bioimpedance signal x(n) does not satisfy the predetermined condition. The filter control unit 60a thereby adjusts the convergence speed of the adaptive filter 30 such that the magnitude of the signal e(n) itself is not reduced too much, while sufficiently reducing the influence of the control signal W(n)×x(n) according to the noise component that appears in the biological information signal e(n).

By performing such control, the biological information providing apparatus 200a of the present embodiment can appropriately follow the convergence speed of the adaptive filter 30 and remove the noise, even when sudden noise is mixed in the bioimpedance signal x(n). Then, when the sudden noise is not mixed, since the step-size parameter is set to a small value, the main signal e(n) can be prevented from being attenuated together with the control signal W(n)×x(n) due to the step-size parameter μ being set to a value that is too large.

After the step-size parameter μ is set to the first value or the second value, the filter control unit 60a may perform the determination again following a predetermined number of cycles of the feedback loop has elapsed, or alternatively following a predetermined period. The step-size parameter μ may be set to a different value according to this determination. The adaptive filter 30 can thereby remove the influence of the noise after increasing the step-size parameter μ according to the sudden noise, and subsequently, reduce the step-size parameter μ when the sudden noise no longer appears.

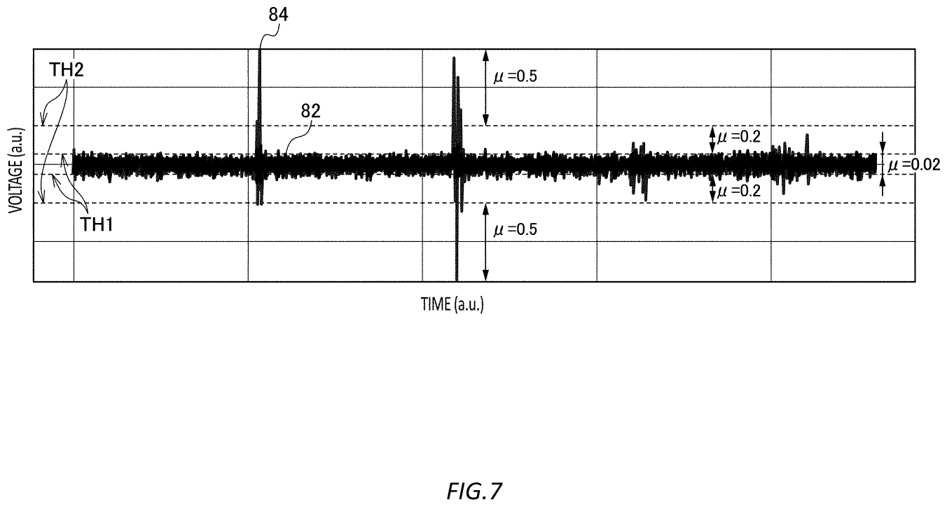

In the present embodiment, the filter control unit 60a performs a determination of whether or not the control signal W(n)×x(n) is equal to or greater than a predetermined magnitude, and switches the convergence speed of the adaptive filter 30 in a two-step manner by changing the step-size parameter μ in a two-step manner. However, the change in the convergence speed of the adaptive filter 30 performed by the filter control unit 60a is not limited to that in a two-step manner. That is, a plurality of thresholds may be provided in the determination of whether or not the control signal W(n)×x(n) is equal to or greater than the predetermined magnitude, and the filter control unit 60a may switch the convergence speed of the adaptive filter 30 in multiple steps according to the magnitude of the noise. This specific example will be described below with reference to FIG. 7.

As described above, when the adaptation algorithm of the adaptive filter 30 is LMS, the filter control unit 60a performs a control to vary the step-size parameter μ. However, the adaptation algorithm of the adaptive filter 30 is not limited to LMS, and the adaptive filter 30 may adopt, as the adaptation algorithm, NLMS, recursive least squares (RLS), Affine projection algorithm (APA). That is, the adaptive filter 30 may be an NLMS adaptive filter, RLS adaptive filter, APA adaptive filter.

In this case, the filter control unit 60a may perform control to set the stability constant ε, together with or alternatively instead of the step-size parameter μ, as a variable parameter from a constant, to dynamically vary the magnitude of the parameter ε. In the formula (1), the convergence speed of the adaptive filter 30 becomes faster when the stability constant ε is reduced as a variable parameter, and the convergence speed of the adaptive filter 30 becomes slower when the stability constant ε is increased as a variable parameter. Therefore, the filter control unit 60a may control the adaptive filter 30 to set the stability constant ε of the adaptive filter 30 to a predetermined first value at the coefficient adjusting unit 34, when the bioimpedance signal x(n) satisfies the predetermined condition. Similarly to the case of adjusting the step-size parameter μ, the filter control unit 60a thereby adjusts the convergence speed of the adaptive filter 30 such that the influence of the control signal W(n)×x(n) according to the noise component that appears in the biological information signal e(n) is reduced. On the other hand, the filter control unit 60a may control the adaptive filter 30 to set the stability constant ε of the adaptive filter 30 to a predetermined second value that is greater than the first value at the coefficient adjusting unit 34, when the bioimpedance signal x(n) does not satisfy the predetermined condition. The filter control unit 60a thereby adjusts the convergence speed of the adaptive filter 30 such that the magnitude of the signal e(n) itself is not reduced too much, while sufficiently reducing the influence of the control signal W(n)×x(n) according to the noise component that appears in the biological information signal e(n).

In another embodiment, the filter control unit 60a may control the variable coefficient filtering unit 32 such that the operation processing speed of the adaptive filter 30 is at a first operation processing speed, when the bioimpedance signal x(n) satisfies the predetermined condition. On the other hand, the filter control unit 60a may control the variable coefficient filtering unit 32 such that the operation processing speed of the adaptive filter 30 is at a second operation processing speed that is slower than the first operation processing speed, when the bioimpedance signal x(n) does not satisfy the predetermined condition. The filter control unit 60a can thereby adjust the convergence speed of the adaptive filter 30, similarly to the adjustment of the step-size parameter μ and the adjustment of the stability constant ε.

For example, when the biological information providing apparatus 200a is implemented in an integrated circuit (IC), each device in the IC is operated based on a working frequency (clock frequency) which refers to a reference clock. As a specific example where the filter control unit 60a changes the operation processing speed of the adaptive filter 30, the filter control unit 60a may change the working frequency of the adaptive filter 30.

That is, the filter control unit 60a may increase the working frequency of the adaptive filter 30 when changing the operation processing speed of the adaptive filter 30 to the first operation processing speed. As an example, the working frequency corresponding to the first operation processing speed may be 6 kHz, or may be 8 kHz. On the other hand, the adaptive filter 30 may reduce the working frequency when changing the operation processing speed of the adaptive filter 30 to the second operation processing speed. In addition, as an example, the working frequency corresponding to the second operation processing speed may be 3 kHz, or may be 2 kHz.

Further, in another embodiment, the filter control unit 60a may control the adaptive filter such that an update interval of the coefficient is at a first interval, when the bioimpedance signal x(n) satisfies the predetermined condition. On the other hand, the filter control unit 60a may control the adaptive filter such that the update interval of the coefficient is at a second interval that is longer than the first interval, when the bioimpedance signal x(n) does not satisfy the predetermined condition. Herein, for example, the second interval may be an interval of a length that is twice the length of the first interval, or may be an interval of a length that is four times the length of the first interval. Alternatively, the filter control unit 60a may perform control to stop the update of the coefficient according to the condition that the bioimpedance signal x(n) satisfies, or may perform control to resume the update of the coefficient for which the update has been stopped. In this case, the filter control unit 60a may perform control such that the update of the coefficient is stopped for a predetermined period and the update of the coefficient is resumed following the predetermined period.

As described in these embodiments, the filter control unit 60a can control the adaptive filter 30 such that the convergence speed of the adaptive filter 30 becomes faster, not only by switching the magnitude of the step-size parameter μ, but also by switching the operation processing speed of the adaptive filter 30 or the update interval of the coefficient. Therefore, at the biological information providing apparatus 200a, the operation processing speed of the adaptive filter 30 or the update interval of its coefficient may be switched, instead of, or alternatively in combination with switching of the magnitude of the step-size parameter μ.

The biological information providing apparatus 200a can switch the convergence speed of the adaptation algorithm at the adaptive filter 30 by any of the approaches listed above. The filter control unit 60a of the biological information providing apparatus 200a may switch the convergence speed of the adaptive filter 30 by selecting any of these approaches, or alternatively by combining these approaches.

It has already been described that the filter control unit 60a may switch the convergence speed of the adaptive filter 30 in multiple steps. In this case, the filter control unit 60a may switch the convergence speed of the adaptive filter 30 in multiple steps by combining a plurality of approached for switching the convergence speed of the adaptive filter 30.

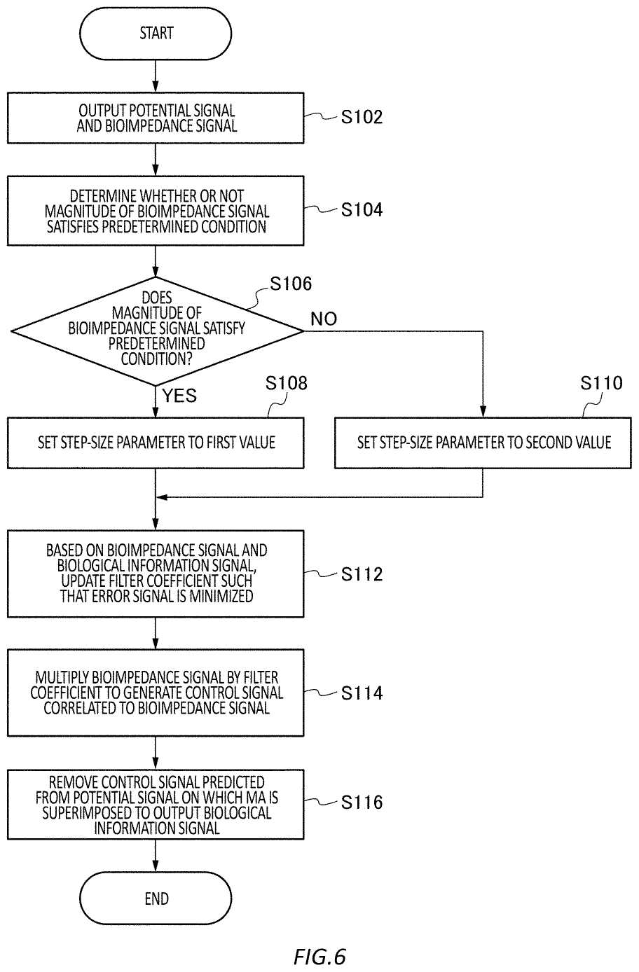

FIG. 6 is an example of a flow diagram showing the processing of the biological information providing apparatus 200a according to the first embodiment. The processing of the biological information providing apparatus 200a of the present embodiment includes steps S102 to S116.

The potential signal measuring unit 10 outputs the potential signal d(n). The bioimpedance signal measuring unit 20 outputs the bioimpedance signal x(n) (S102). The bioimpedance signal determination unit 62a of the filter control unit 60a determines whether or not the magnitude of the bioimpedance signal x(n) satisfies the predetermined condition (S104).

The processing is branched depending on whether or not the magnitude of the bioimpedance signal x(n) satisfies the predetermined condition (S106). When the magnitude of the bioimpedance signal x(n) satisfies the predetermined condition, the processing is advanced to S108, and when the magnitude of the bioimpedance signal x(n) does not satisfy the predetermined condition, the processing is advanced to S110.

The step size setting unit 64 of the filter control unit 60a sets the step-size parameter μ of the adaptive filter 30 to a first value (for example, μ=0.8) (S108). Subsequently, the processing is advanced to S112.

The step size setting unit 64 of the filter control unit 60a sets the step-size parameter μ of the adaptive filter 30 to a second value (for example, μ=0.2) that is smaller than the first value (S110). Subsequently, the processing is advanced to S112 and is converged.

The coefficient adjusting unit 34 updates the coefficient W(n) such that an error signal H(n)×x(n)−W(n)×x (n) is minimized, based on the bioimpedance signal x(n) and the biological information signal e(n) (S112). The adaptive filter 30 multiplies the bioimpedance signal by the coefficient W(n) to generate a control signal W(n)×x(n) that is correlated to the bioimpedance signal (S114).

The subtractor 42 of the biological information signal output unit 40 removes the control signal W(n)×x(n) predicted from the potential signal d(n) on which the motion artifact (MA) is superimposed. The biological information signal output unit 40 thereby outputs the biological information signal e(n) from which the control signal W(n)×x(n) is removed (S116). The processing is terminated by the above.

Through such processing, even when sudden noise is mixed in the measurement of the potential signal d(n), the convergence speed of the adaptive filter 30 for removing the noise can be appropriately followed, and the main signal e(n) can be prevented from being attenuated together with the control signal W(n)×x(n) due to an excessive coefficient updating frequency.

FIG. 7 illustrates an example of a graph in a case where switching among a plurality of step-size parameters μ is performed according to a plurality of thresholds. In the figure, an example where two thresholds indicating a threshold TH1 and a threshold TH2 are provided as thresholds in the magnitude of the bioimpedance signal x(n) is illustrated. Also in FIG. 7, similarly to FIG. 4A and FIG. 4B, a polygonal line 84 indicating the potential signal d(n) and a polygonal line 82 indicating the biological information signal e(n) after the noise is removed by the adaptive filter 30 are illustrated.

The threshold TH1 and the threshold TH2 are predetermined thresholds, and the threshold TH2 is smaller than the threshold TH1. In this example, the step-size parameter is switched in three steps by ranges defined according to the threshold TH1 and the threshold TH2.

The bioimpedance signal determination unit 62a determines whether the magnitude of the bioimpedance signal is in a range that is greater than the threshold TH1, in a range that is greater than the threshold TH2 and equal to or less than the threshold TH1, or in a range that is equal to or less than the threshold TH2. As an example, the threshold TH1 is 1 mV, and the threshold TH2 is 0.25 mV.

The step size setting unit 64 controls the coefficient adjusting unit 34 to set the step-size parameter μ to 0.5, when the magnitude of the bioimpedance signal is in the range that is greater than the threshold TH1.

The step size setting unit 64 controls the coefficient adjusting unit 34 to set the step-size parameter μ to 0.2, when the magnitude of the bioimpedance signal is in the range that is greater than the threshold TH1.

The filter control unit 60a may thereby control the coefficient adjusting unit 34 to set the step-size parameter μ of the coefficient adjusting unit 34 to 0.5, when the magnitude of the bioimpedance signal x(n) exceeds TH1. In addition, the filter control unit 60a may control the coefficient adjusting unit 34 to set the step-size parameter μ of the coefficient adjusting unit 34 to 0.2, when the magnitude of the bioimpedance signal x(n) is equal to or less than TH1 and exceeds TH2. In addition, the filter control unit 60a may control the coefficient adjusting unit 34 to set the step-size parameter μ of the coefficient adjusting unit 34 to 0.02, when the magnitude of the bioimpedance signal x(n) is equal to or less than TH2.

In the present embodiment, the threshold TH1 is an example of the “first threshold value”, and the threshold TH2 is an example of the “second threshold value”. In addition, the step-size parameter μ=0.5 is an example of the “first value”, the step-size parameter μ=0.2 is an example of the “second value”, and the step-size parameter μ=0.02 is an example of the “third value”. Note that, more thresholds may be set than in the present embodiment, and accordingly, the step-size parameter μ may be switched in even more steps than in the present embodiment.

In this manner, with the filter control unit 60a switching the step-size parameter μ in three steps, the noise mixed in the potential signal d(n) is reduced with an appropriate accuracy. Furthermore, in a range where the potential signal d(n) is small, since the step-size parameter μ is also set to be small, the main signal e(n) is prevented from being attenuated together with the control signal W(n)×x(n).

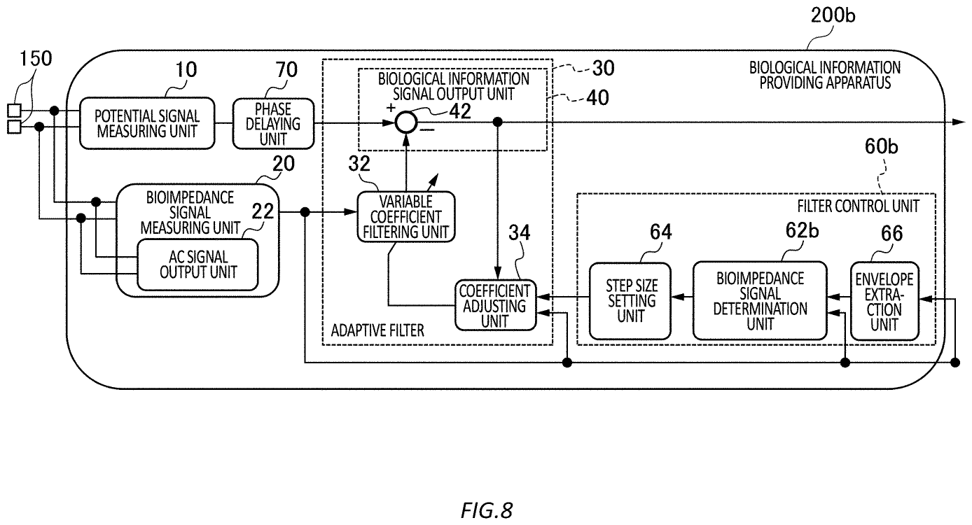

FIG. 8 illustrates an example of a block diagram of a configuration of a biological information providing apparatus 200b according to a second embodiment. Hereinafter, the configuration of the biological information providing apparatus 200b will be described focusing on a difference thereof from the biological information providing apparatus 200a. The biological information providing apparatus 200b includes a filter control unit 60b instead of the filter control unit 60a, relative to the components included in the biological information providing apparatus 200a. In addition, the biological information providing apparatus 200b includes a phase delaying unit 70 between the potential signal measuring unit 10 and the biological information signal output unit 40.

The filter control unit 60b controls the adaptive filter 30 such that the convergence speed of the adaptive filter 30 is faster in a case where the bioimpedance signal x(n) satisfies a predetermined condition than in a case where the bioimpedance signal x(n) does not satisfy the condition. The filter control unit 60b includes an envelope extraction unit 66, a bioimpedance signal determination unit 62b, and a step size setting unit 64.

The envelope extraction unit 66 performs envelope extraction on the bioimpedance signal x(n) and outputs a signal indicating a contour of the sudden noise. The envelope extraction performed by the envelope extraction unit 66 includes absolute value conversion of the bioimpedance signal x(n) and low pass filtering processing. Herein, the absolute value conversion of the signal value may be performed by squaring the signal value, as an example. The processing performed on the bioimpedance signal x(n) by the envelope extraction unit 66 may include amplification of a signal after the low pass filtering, arithmetic processing such as addition of a predetermined constant to the signal value. The envelope extraction unit 66 thereby outputs a signal indicating a contour of the sudden noise. This signal indicating the contour of the waveform is an example of the “reference waveform signal”. Through such envelope extraction, the filter control unit 60b can easily extract only the noise that is suddenly generated by the motion artifact or the like from the bioimpedance signal x(n). As an example, the envelope extraction unit 66 includes a low-pass filter having a cutoff frequency of 10 Hz.

Herein, the envelope extraction unit 66 performing envelope extraction including low pass filtering on the bioimpedance signal x(n) may cause a signal delay in the control signal output by the variable coefficient filtering unit 32, relative to the signal obtained by the potential signal measuring unit 10. For example, when a low-pass filter having a cutoff frequency of 10 Hz is used as the low-pass filter included in the envelope extraction unit 66, a signal delay of about 30 milliseconds occurs. When such a signal delay occurs, by adding the phase delaying unit 70, even when the envelope extraction unit 66 is used, a phase can be adjusted between the signal output by the potential signal measuring unit 10 and the signal output by the variable coefficient filtering unit 32.

The bioimpedance signal determination unit 62b determines the presence or absence of a mixture of the sudden noise from the contour of the bioimpedance signal x(n) through the envelope extraction unit 66. In particular, the bioimpedance signal determination unit 62b determines whether or not the magnitude of the bioimpedance signal x(n) exceeds the magnitude of the reference waveform signal, which is a signal obtained by performing filtering processing on the bioimpedance signal x(n) by the envelope extraction unit 66.

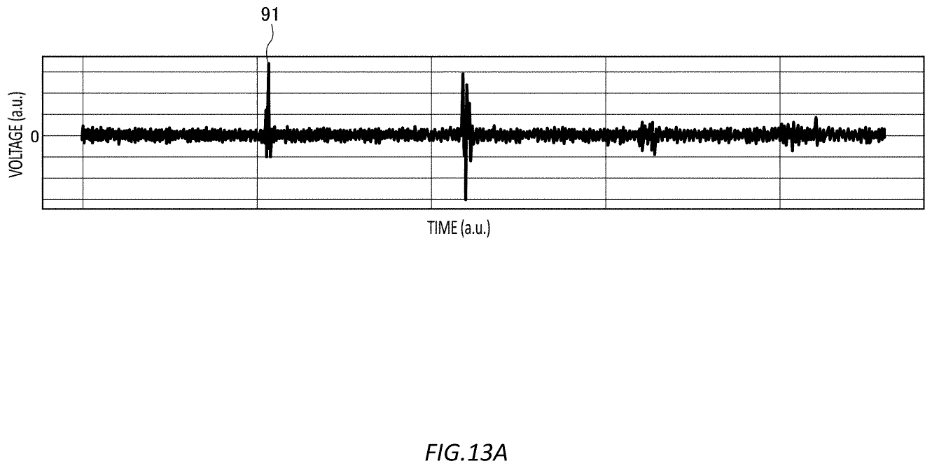

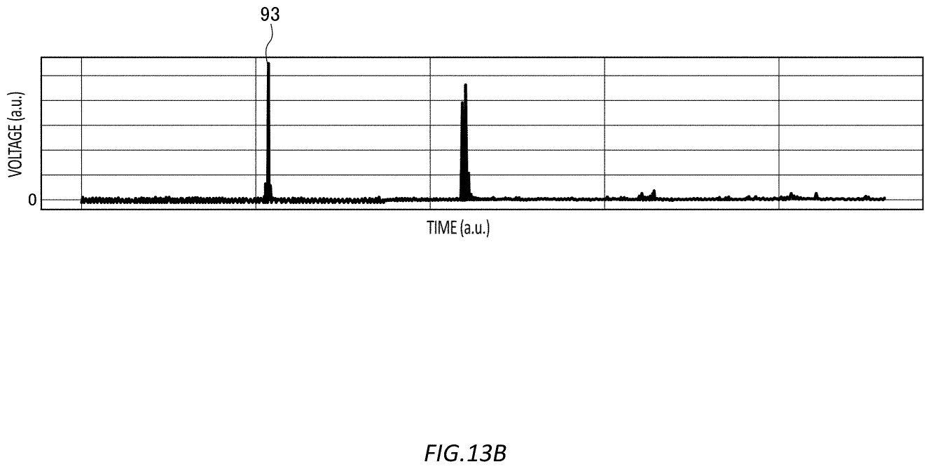

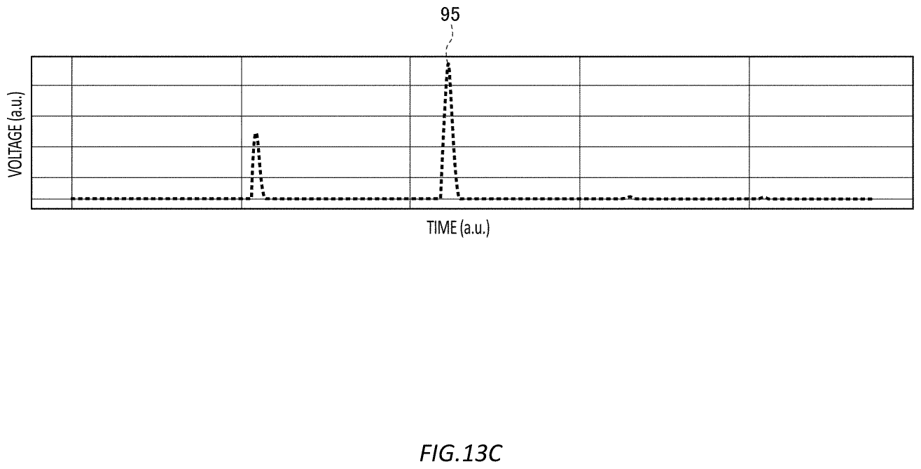

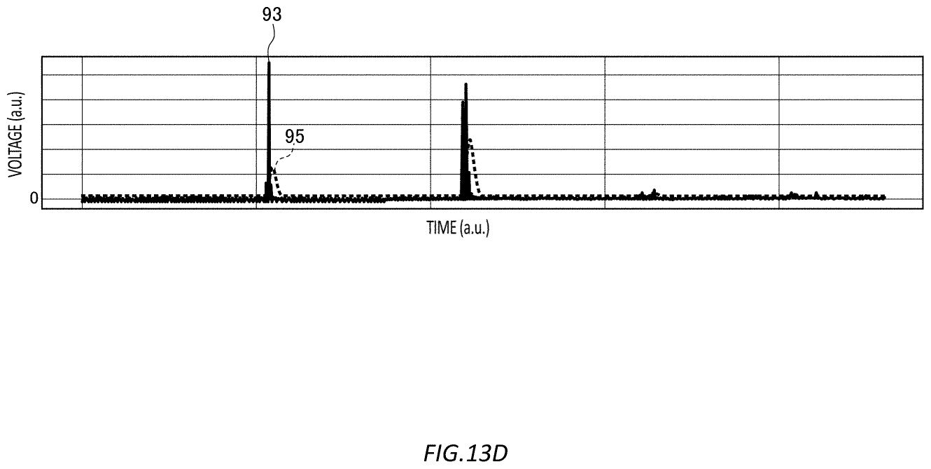

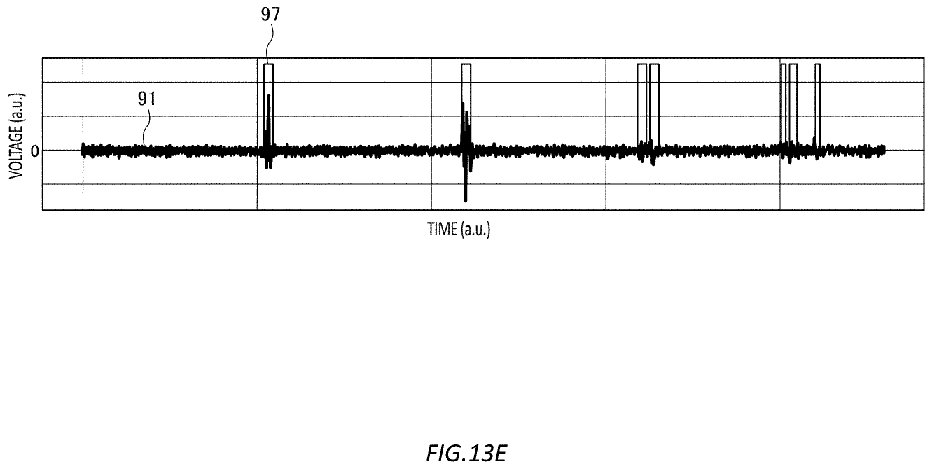

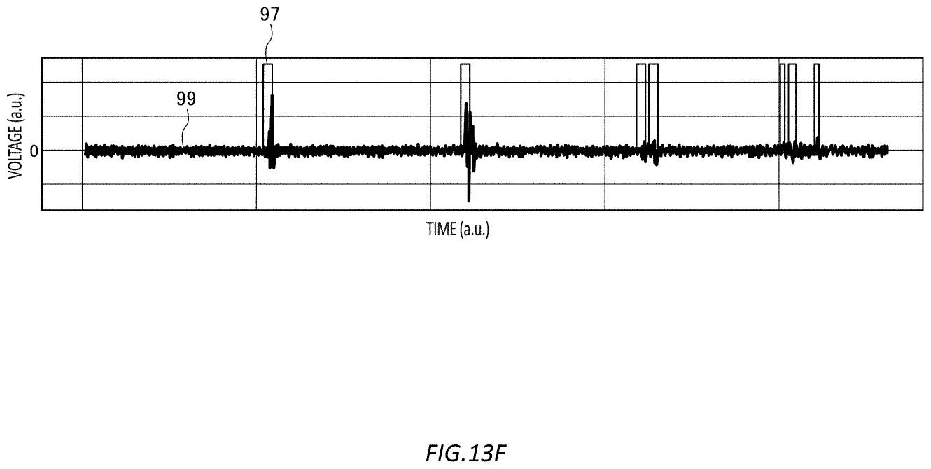

The filter control unit 60b thereby determines that the bioimpedance signal x(n) satisfies the predetermined condition, when the magnitude of the bioimpedance signal x(n) exceeds the magnitude of the reference waveform signal obtained by performing filtering processing on the bioimpedance signal x(n) by the envelope extraction unit 66. Note that, this processing is equivalent to the processing in which the processing described below for the potential signal d(n) in the fourth embodiment with reference to FIG. 13A to FIG. 13F is replaced with the processing performed on the bioimpedance signal x(n).

Herein, the step size setting unit 64 sets the magnitude of the step-size parameter μ based on a determination result of the potential signal determination unit 67b. The functionality of the step size setting unit 64 is similar to that of the step size setting unit 64 included in the filter control unit 60a, except that it is based on the determination result of the bioimpedance signal determination unit 62d.

The phase delaying unit 70 delays the phase of the potential signal d(n) output from the potential signal measuring unit 10 relative to the control signal W(n)×x(n) output from the variable coefficient filtering unit 32 to adjust the phase. The phase delaying unit 70 thereby delays the phase of the potential signal d(n) input to the biological information signal output unit 40 by a delay time due to the filtering processing by the envelope extraction unit 66. Note that, when it is considered that the phase delay by the envelope extraction unit 66 is not large at the biological information providing apparatus 200b of the present embodiment, the phase delaying unit 70 can be omitted.

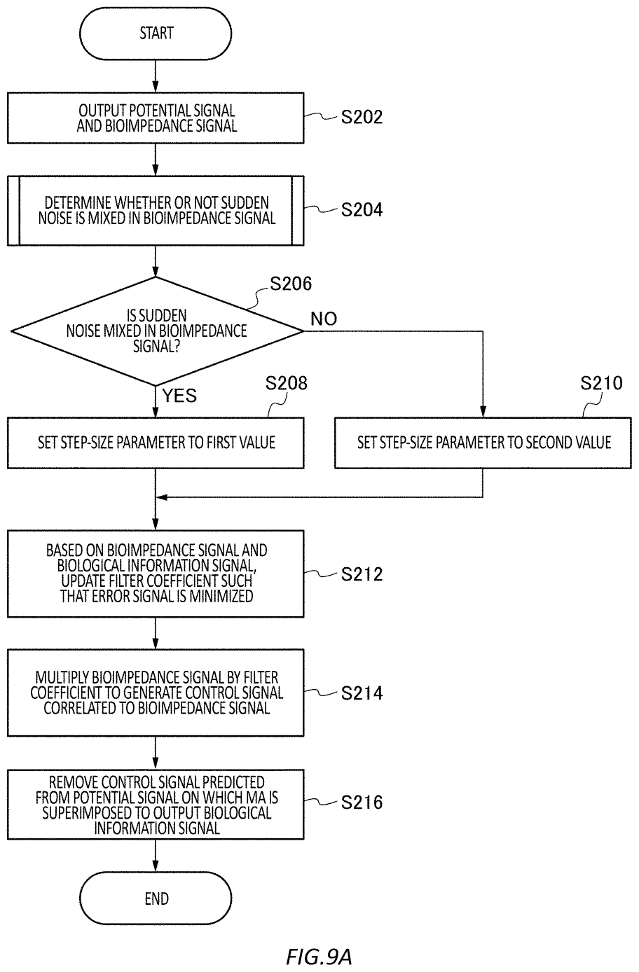

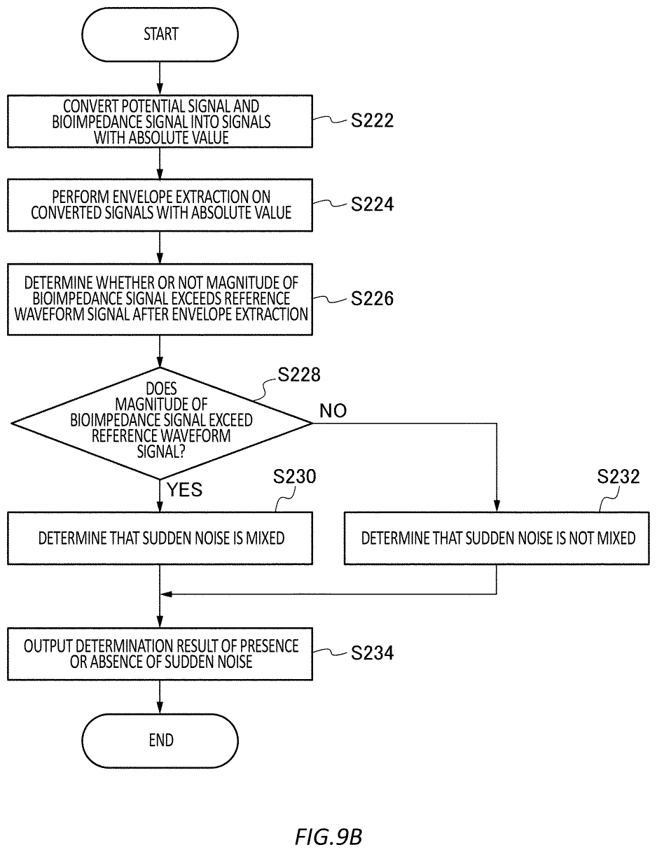

FIG. 9A is an example of a flow diagram showing the processing of the biological information providing apparatus 200b according to the second embodiment. The processing of the present embodiment includes steps S202 to S216.

The potential signal measuring unit 10 outputs the potential signal d(n). The bioimpedance signal measuring unit 20 outputs the bioimpedance signal x(n) (S202).

The bioimpedance signal determination unit 62b of the filter control unit 60 determines whether or not the sudden noise is mixed in the bioimpedance signal x(n) (S204). This step will be detailed later with reference to FIG. 9B.

The processing is branched depending on whether or not the sudden noise is determined to be mixed in the bioimpedance signal (S206). When it is determined that the sudden noise is mixed in the bioimpedance signal x(n), the processing is advanced to S208, and when it is not determined that the sudden noise is mixed in the bioimpedance signal x(n), the processing is advanced to S210.

The step size setting unit 64 of the filter control unit 60b sets the step-size parameter to a first value (S208). Subsequently, the processing is advanced to S212.

The step size setting unit 64 of the filter control unit 60b sets the step-size parameter to a second value that is smaller than the first value (S210). Subsequently, the processing is advanced to S212 and is converged.

The coefficient adjusting unit 34 updates the coefficient W(n) such that an error signal H(n)×x(n)−W(n)×x (n) is minimized, based on the bioimpedance signal x(n) and the biological information signal e(n) (S212). The variable coefficient filtering unit 32 multiplies the bioimpedance signal x(n) by the coefficient W(n) to generate a control signal W(n)×x(n) that is correlated to the bioimpedance signal x(n) (S214).

The subtractor 42 of the biological information signal output unit 40 removes the control signal W(n)×x(n) predicted from the potential signal d(n) on which the motion artifact is superimposed. The biological information signal output unit 40 thereby outputs the biological information signal e(n) from which the control signal W(n)×x(n) is removed (S216). The processing is terminated by the above.

FIG. 9B is an example of a flow diagram further detailing step S204 of FIG. 9A. The processing of the present embodiment includes steps S222 to S234.

The filter control unit 60b converts the potential signal d(n) and the bioimpedance signal x(n) into signals with an absolute value (S222). For this purpose, the filter control unit 60b may have a modulator, a mixer, and/or arithmetic unit (not shown) or the like.

The envelope extraction unit 66 performs envelope extraction including absolute value conversion and low pass filtering on the signal with an absolute value obtained through the absolute value conversion, to obtain a reference waveform signal (S224). The bioimpedance signal determination unit 62b determines whether or not the bioimpedance signal exceeds the magnitude of the reference waveform signal after the envelope extraction including the absolute value conversion and the low pass filtering by the envelope extraction unit 66 (S226).

The processing is branched depending on whether or not the bioimpedance signal exceeds the magnitude of the reference waveform signal after the envelope extraction including low pass filtering by the envelope extraction unit 66 (S228). When the bioimpedance signal exceeds the magnitude of the reference waveform signal, the processing is advanced to S230, and when the bioimpedance signal does not exceed the magnitude of the reference waveform signal, the processing is advanced to S232.

When the bioimpedance signal exceeds the magnitude of the reference waveform signal, the bioimpedance signal determination unit 62b determines that the sudden noise is mixed (S230). Subsequently, the processing is advanced to S234.

When the bioimpedance signal does not exceed the magnitude of the reference waveform signal, the bioimpedance signal determination unit 62b determines that the sudden noise is not mixed (S232). Subsequently, the processing is advanced to S234.

The bioimpedance signal determination unit 62b outputs a determination result of the presence or absence of the sudden noise (S234). The processing of S204 is terminated by the above.

Through such processing, also with the biological information providing apparatus 200b, even when sudden noise is mixed in the measurement of the biological information signal e(n), the convergence speed of the adaptive filter 30 to remove the noise can be appropriately followed. Furthermore, the main signal e(n) can be prevented from being attenuated together with the control signal W(n)×x(n) due to an excessive coefficient updating frequency.

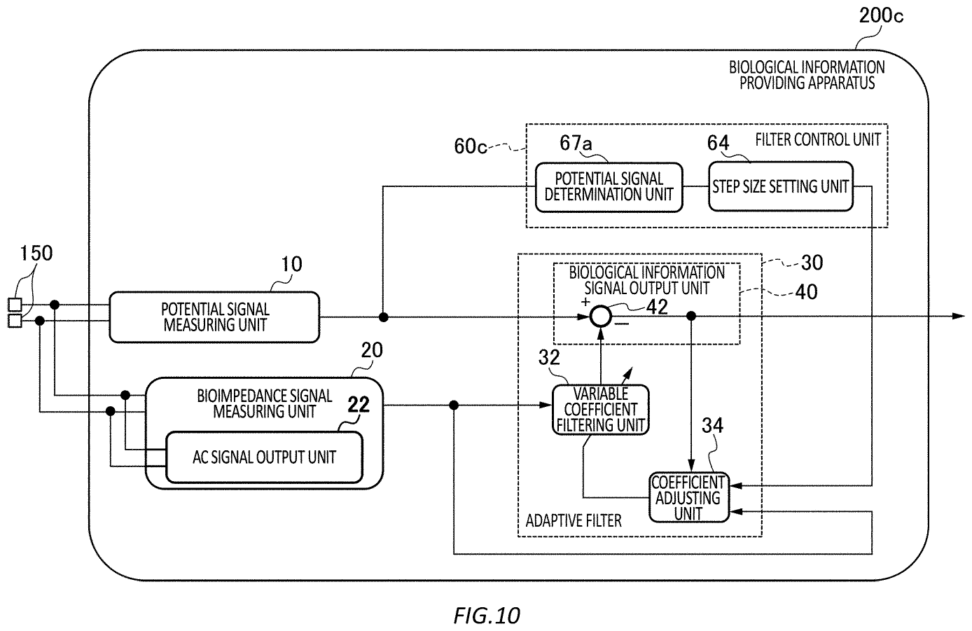

FIG. 10 illustrates an example of a block diagram of a configuration of a biological information providing apparatus 200c according to a third embodiment. Hereinafter, the configuration of the biological information providing apparatus 200c will be described focusing on a difference thereof from the biological information providing apparatus 200a. The biological information providing apparatus 200c includes a filter control unit 60c instead of the filter control unit 60a, relative to the components included in the biological information providing apparatus 200a. Unlike the filter control unit 60a, the filter control unit 60c is connected to an output of the potential signal measuring unit 10, instead of the output of the bioimpedance signal measuring unit 20.

The filter control unit 60c controls the coefficient adjusting unit 34 such that the convergence speed of the adaptive filter 30 is faster in a case where the potential signal d(n) satisfies the predetermined condition than in a case where the potential signal d(n) does not satisfy the condition. As described for FIG. 3, the component x(n) also appears in the potential signal d(n)=s(n)+H(n)×x(n). Therefore, the noise x(n) associated with the variation in the bioimpedance BioZ can also be detected by measuring the potential signal d(n). The filter control unit 60c includes a potential signal determination unit 67a and the step size setting unit 64.

The potential signal determination unit 67a determines whether or not the potential signal d(n) satisfies a predetermined condition. As an example, the filter control unit 60c determines that the potential signal d(n) satisfies the predetermined condition when the magnitude of the potential signal d(n) exceeds a predetermined first threshold value.

Note that, also for the potential signal d, similarly to the case of the bioimpedance signal x(n) in the first embodiment, a plurality of thresholds may be provided. The filter control unit 60 may thereby switch the step-size parameter μ in multiple steps.

Therefore, the filter control unit 60c may control the coefficient adjusting unit 34 to set the step-size parameter μ of the coefficient adjusting unit 34 to a predetermined first value (for example, μ=0.5), when the magnitude of the potential signal d(n) exceeds the first threshold value. In addition, the filter control unit 60c may control the coefficient adjusting unit 34 to set the step-size parameter μ of the coefficient adjusting unit 34 to a second value (for example, μ=0.2) that is smaller than the first value, when the magnitude of the bioimpedance signal x(n) is equal to or less than TH1 and exceeds TH2. In addition, the filter control unit 60c may control the coefficient adjusting unit 34 to set the step-size parameter μ of the coefficient adjusting unit 34 to a third value (for example, μ=0.02) that is smaller than the second value, when the magnitude of the bioimpedance signal x(n) is equal to or less than TH2.

Also in the present embodiment, the adaptive filter 30 may adopt NLMS as the adaptation algorithm. That is, the adaptive filter 30 may be an NLMS adaptive filter.

In this case the filter control unit 60c may perform control of the stability constant ε, together with the step-size parameter μ, or alternatively instead of p. Therefore, the filter control unit 60c may control the coefficient adjusting unit 34 to set the stability constant ε of the adaptive filter 30 to a predetermined first value, when the potential signal d(n) satisfies the predetermined condition. On the other hand, the filter control unit 60c may control the coefficient adjusting unit 34 to set the stability constant ε of the adaptive filter 30 to a predetermined second value that is greater than the first value, when the potential signal d(n) does not satisfy the predetermined condition.

In another embodiment, the filter control unit 60c may control the coefficient adjusting unit 34 such that the operation processing speed of the adaptive filter 30 is at a first operation processing speed, when the potential signal d(n) satisfies the predetermined condition. On the other hand, the filter control unit 60c may control the coefficient adjusting unit 34 such that the operation processing speed of the adaptive filter 30 is at a second operation processing speed that is slower than the first operation processing speed, when the potential signal d(n) does not satisfy the condition.

Further, in another embodiment, the filter control unit 60c may control the coefficient adjusting unit 34 such that an update interval of the coefficient is at a first interval, when the potential signal d(n) satisfies the predetermined condition. On the other hand, the filter control unit 60c may control the coefficient adjusting unit 34 such that the update interval of the coefficient is at a second interval that is longer than the first interval, when the potential signal d(n) does not satisfy the condition.

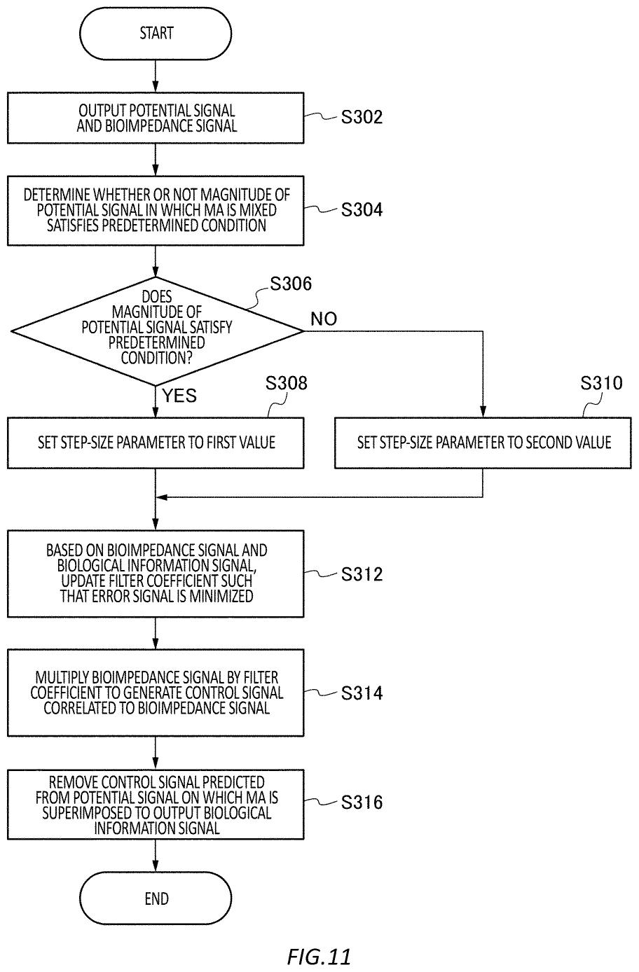

FIG. 11 is an example of a flow diagram showing the processing of the biological information providing apparatus 200c according to the third embodiment. The processing of the biological information providing apparatus 200c of the present embodiment includes steps S302 to S316.

The potential signal measuring unit 10 outputs the potential signal d(n). The bioimpedance signal measuring unit 20 outputs the bioimpedance signal x(n) (S302). The potential signal determination unit 67 of the filter control unit 60c determines whether or not the magnitude of the potential signal d(n) satisfies a predetermined condition (S304).

The processing is branched depending on whether or not the magnitude of the potential signal d(n) satisfies the predetermined condition (S306). When the magnitude of the potential signal d(n) exceeds the predetermined threshold, the processing is advanced to S308, and when the magnitude of the potential signal d(n) does not exceed the predetermined threshold, the processing is advanced to S310.

The step size setting unit 64 of the filter control unit 60c sets the step-size parameter μ of the adaptive filter 30 to a first value (S308). Subsequently, the processing is advanced to S312.

The step size setting unit 64 of the filter control unit 60c sets the step-size parameter μ of the adaptive filter 30 to a second value that is smaller than the first value (S310). Subsequently, the processing is advanced to S312 and is converged.

The coefficient adjusting unit 34 updates the coefficient W(n) such that an error signal H(n)×x(n)−W(n)×x (n) is minimized, based on the bioimpedance signal x(n) and the biological information signal e(n) (S312). The variable coefficient filtering unit 32 multiplies the bioimpedance signal x(n) by the coefficient W(n) to generate a control signal W(n)×x(n) that is correlated to the bioimpedance signal x(n) (S314).

The subtractor 42 of the biological information signal output unit 40 removes the control signal W(n)×x(n) predicted from the potential signal d(n) on which the motion artifact is superimposed. The biological information signal output unit 40 thereby outputs the biological information signal e(n) from which the control signal W(n)×x(n) is removed (S316). The processing is terminated by the above.

Through such processing, also with the biological information providing apparatus 200c, even when sudden noise is mixed in the measurement of the biological information signal e(n), the convergence speed of the adaptive filter 30 to remove the noise can be appropriately followed. Furthermore, the main signal e(n) can be prevented from being attenuated together with the control signal W(n)×x(n) due to an excessive coefficient updating frequency.

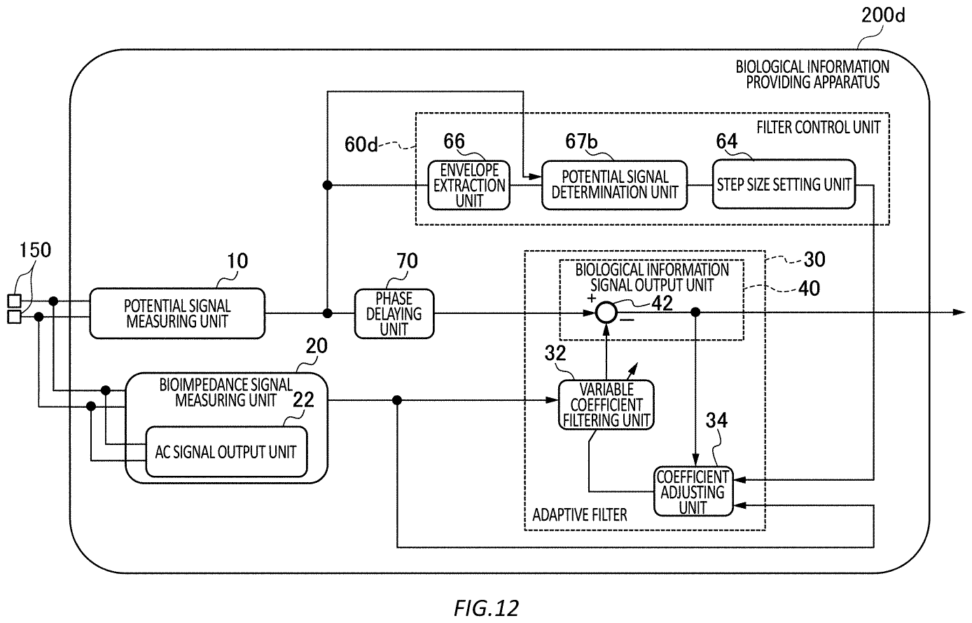

FIG. 12 illustrates an example of a block diagram of a configuration of a biological information providing apparatus 200d according to a fourth embodiment. Hereinafter, the configuration of the biological information providing apparatus 200d will be described focusing on a difference thereof from the biological information providing apparatus 200a. Unlike the biological information providing apparatus 200a, the biological information providing apparatus 200d includes a filter control unit 60d. In addition, the biological information providing apparatus 200d includes a phase delaying unit 70 between the potential signal measuring unit 10 and the biological information signal output unit 40, similarly to the biological information providing apparatus 200b.

The filter control unit 60d controls the adaptive filter 30 such that the convergence speed of the adaptive filter 30 is faster in a case where the potential signal d(n) satisfies a condition indicating that the control signal W(n)×x(n) is equal to or greater than a predetermined magnitude than in a case where the potential signal d(n) does not satisfy the condition. The filter control unit 60d includes the envelope extraction unit 66, the potential signal determination unit 67b, and the step size setting unit 64.

The envelope extraction unit 66 filters the potential signal d(n) and outputs a signal indicating a contour of the sudden noise. The filter control unit 60d can thereby easily extract only the noise that is suddenly generated by the motion artifact or the like from the potential signal d(n). The envelope extraction unit 66 of the filter control unit 60d act similarly to the envelope extraction unit 66 of the filter control unit 60b, except that it performs envelope extraction on the potential signal d(n).

The potential signal determination unit 67b determines the presence or absence of mixture of the sudden noise from the contour of the potential signal d(n) through the envelope extraction unit 66. In particular, the potential signal determination unit 67b determines whether or not the magnitude of the potential signal d(n) through the envelope extraction unit 66 exceeds the magnitude of the signal obtained by filtering the potential signal d(n) by the envelope extraction unit 66. The filter control unit 60c thereby determines that the potential signal d(n) satisfies the predetermined condition and that the control signal W(n)×x(n) is increased in the potential signal d(n), when the magnitude of the potential signal d(n) exceeds the magnitude of the reference waveform signal obtained by performing envelope extraction on the potential signal d(n) by the envelope extraction unit 66. In the case of the present embodiment, unlike the second embodiment, the signal obtained by performing envelope extraction on the potential signal d(n) by the envelope extraction unit 66 is equivalent to the “reference waveform signal”.

The phase delaying unit 70 delays the phase of the potential signal d(n) output from the potential signal measuring unit 10 relative to the control signal W(n)×x(n) output from the variable coefficient filtering unit 32 to adjust the phase. The phase delaying unit 70 thereby delays the phase of the potential signal d(n) input to the biological information signal output unit 40 by a delay time due to the filtering processing by the envelope extraction unit 66. Note that, also in the biological information providing apparatus 200d of the present embodiment, when it is considered that the phase delay by the envelope extraction unit 66 is not large, the phase delaying unit 70 can be omitted.

FIG. 13A is an example of a graph for describing the functionality of a filter control unit 60d. The signal shown in the figure is an example of a graph of the potential signal d(n) output by the biological information signal output unit 40. The polygonal line 91 indicates the potential signal d(n) input to the filter control unit 60d. Herein, similarly to FIG. 4A or the like, in the graph, the unit of each axis is shown by an arbitrary unit.

FIG. 13B is an example of a graph for describing the functionality of the filter control unit 60d. The signal shown in the figure is a graph obtained by performing absolute value conversion on the power, relative to the graph in FIG. 13A, and is indicated by the polygonal line 93. Herein, the absolute value conversion may be performed by squaring the signal value. Herein, in FIG. 13B to FIG. 13F, not only are the unit of each axis indicated by the arbitrary unit, but the graphs having scales that are different from FIG. 13A in the vertical axis are illustrated.

In the graph in FIG. 13A, the voltage showed both positive and negative values. In FIG. 13B, these positive and negative values are shown as absolute values by squaring them for each time. Therefore, when the noise is mixed, the peak of the noise component appears large, and the contribution of components at another time appears small.

FIG. 13C is an example of a graph for describing the functionality of the filter control unit 60d. The envelope extraction unit 66 acts on the graph of absolute values obtained in FIG. 13B, and performs envelope extraction to obtain a contour of the noise. The signal indicating the contour of the waveform obtained by performing the envelope extraction is illustrated by the polygonal line 95. The signal indicated by the polygonal line 95 is equivalent to the “reference waveform signal”.

FIG. 13D is an example of a graph for describing the functionality of the filter control unit 60d. The bioimpedance signal determination unit 62b determines whether or not the signal in FIG. 13B is greater than the reference waveform signal.

FIG. 13E is an example of a graph for describing the functionality of the filter control unit 60d. In the figure, the bioimpedance signal x(n) similar to that in FIG. 13A, and frames 97 indicating noise mixture positions are illustrated. This frame indicating the noise mixture position is illustrated at a position delayed by the processing time of the filtering processing, due to the envelope extraction FIG. 13B being performed.

As an example, this delay time is about 30 milliseconds. The phase delaying unit 70 delays the phase of the potential signal d(n) by a delay time due to the envelope extraction including the low pass filtering, which allows the signal of the noise mixture portion to fit well in the frame 97.

FIG. 13F is an example of a graph for describing the functionality of the filter control unit 60d. In the figure, a graph obtained by actually delaying the potential signal d(n) by the time from the graph in FIG. 13D is illustrated by the polygonal line 99. It can be read that the noise mixture location in the potential signal d(n) is well fit in the location indicated within the frame.

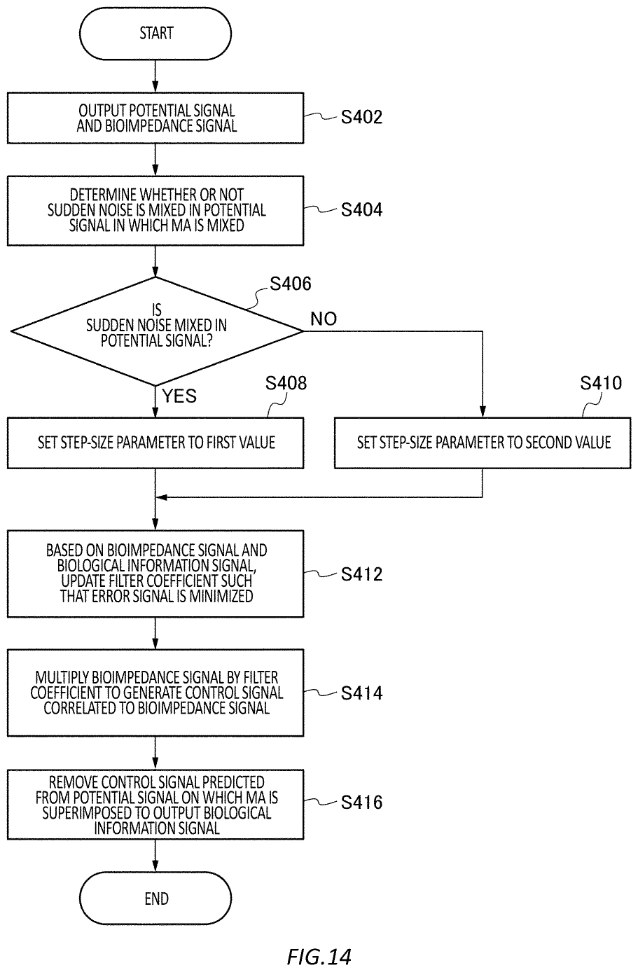

FIG. 14 is an example of a flow diagram showing the processing of the biological information providing apparatus 200d according to the fourth embodiment. The processing of the biological information providing apparatus 200d in the present embodiment includes steps S402 to S416.

The potential signal measuring unit 10 outputs the potential signal d(n). The bioimpedance signal measuring unit 20 outputs the bioimpedance signal x(n) (S402).

The potential signal determination unit 67b of the filter control unit 60 determines whether or not sudden noise is mixed in the bioimpedance signal x(n) (S404). Although this step may be subdivided as in FIG. 9B, since a similar description is made by replacing the description for the bioimpedance signal x(n) with the potential signal d(n), description for the biological information providing apparatus 200d will be omitted.

The processing is branched depending on whether or not it is determined that the sudden noise is mixed in the potential signal d(n) (S406). When it is determined that the sudden noise is mixed in the potential signal d(n), the processing is advanced to S408, and when it is not determined that the sudden noise is mixed in the potential signal d (n), the processing is advanced to S410.

The step size setting unit 64 of the filter control unit 60d sets the step-size parameter to a first value (S408). Subsequently, the processing is advanced to S412.

The step size setting unit 64 of the filter control unit 60b sets the step-size parameter to a second value that is smaller than the first value (S410). Subsequently, the processing is advanced to S412 and is converged.

The coefficient adjusting unit 34 updates the coefficient W(n) such that an error signal H(n)×x(n)−W(n)×x (n) is minimized, based on the bioimpedance signal x(n) and the biological information signal e(n) (S412). The variable coefficient filtering unit 32 multiplies the bioimpedance signal x(n) by the coefficient W(n) to generate a control signal W(n)×x(n) that is correlated to the bioimpedance signal x(n) (S414).

The subtractor 42 of the biological information signal output unit 40 removes the control signal W(n)×x(n) predicted from the potential signal d(n) on which the motion artifact is superimposed. The biological information signal output unit 40 thereby outputs the biological information signal e(n) from which the control signal W(n)×x(n) is removed (S416).

Through such processing, also with the biological information providing apparatus 200d, even when sudden noise is mixed in the measurement of the biological information signal e(n), the convergence speed of the adaptive filter 30 to remove the noise can be appropriately followed. Furthermore, the main signal e(n) can be prevented from being attenuated together with the control signal W(n)×x(n) due to an excessive coefficient updating frequency.

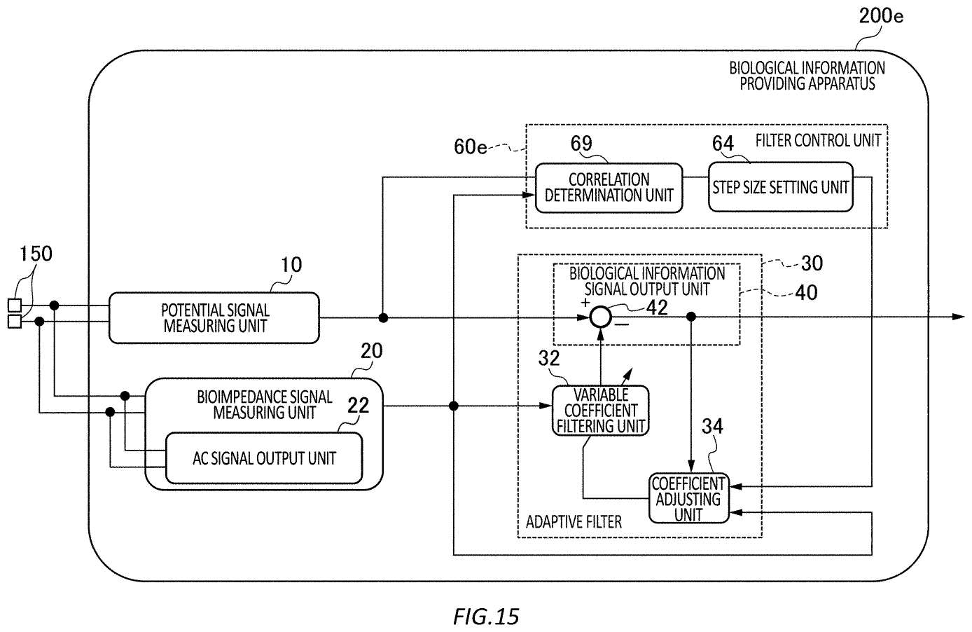

FIG. 15 illustrates an example of a block diagram of a configuration of a biological information providing apparatus 200e according to a fifth embodiment. Hereinafter, the configuration of the biological information providing apparatus 200e will be described focusing on a difference thereof from the biological information providing apparatus 200a. Unlike the biological information providing apparatus 200a, the biological information providing apparatus 200e includes a filter control unit 60e.

The filter control unit 60e includes a correlation determination unit 69 and the step size setting unit 64. Furthermore, unlike the filter control units 60a to 60d, the filter control unit 60e is connected to both outputs of the potential signal measuring unit 10 and the bioimpedance signal measuring unit 20.

The correlation determination unit 69 compares the potential signal d(n) with the bioimpedance signal x(n). For example, the correlation determination unit 69 may perform windowed Fourier transform by fast Fourier transform (FFT) or the like on the potential signal d(n) and the bioimpedance signal x(n) during a time window of a predetermined period, and compare frequency components. Then, when the noise correlated to each of the potential signal d(n) and the bioimpedance signal x(n) appears, and also the similarity between the noise exceeds a predetermined threshold, the correlation determination unit 69 may determine that the magnitude of the control signal W(n)×x(n) indicating the noise component included in the potential signal d(n) satisfies a predetermined condition. The filter control unit 60e may thereby determine that the potential signal d(n) or the bioimpedance signal x(n) satisfies the predetermined condition, when the degree of correlation between the potential signal d(n) and the bioimpedance signal x(n) exceeds the predetermined threshold.

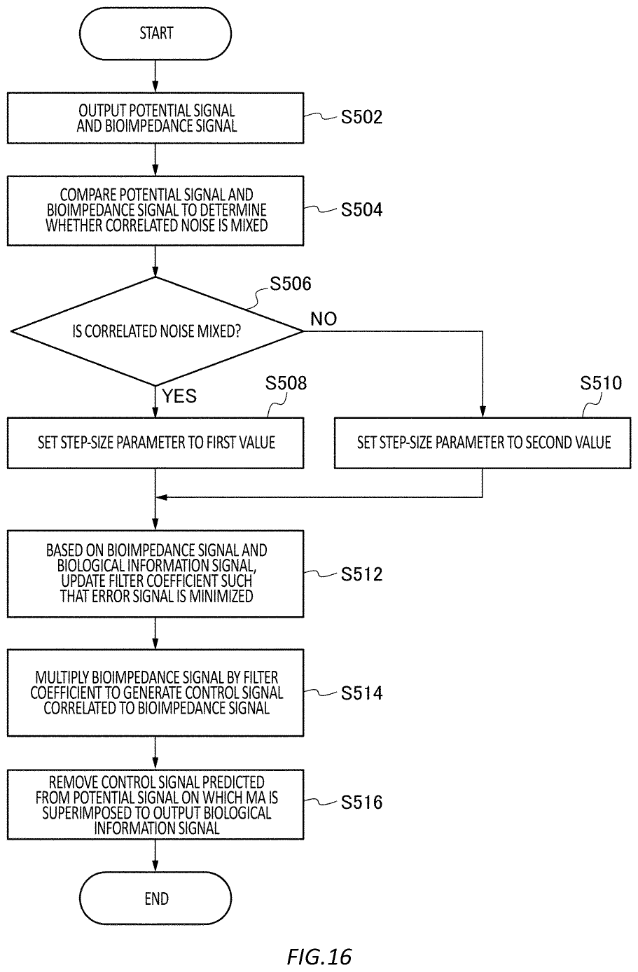

FIG. 16 is an example of a flow diagram showing processing of the biological information providing apparatus 200e according to the fifth embodiment. The processing of the biological information providing apparatus 200e in the fifth embodiment includes steps S502 to S516.

The potential signal measuring unit 10 outputs the potential signal d(n). The bioimpedance signal measuring unit 20 outputs the bioimpedance signal x(n) (S502).

The correlation determination unit 69 of the filter control unit 60e compares the potential signal d(n) with the bioimpedance signal x(n), and determines whether or not noise correlated to the noise that appears in the bioimpedance signal x is mixed in the potential signal d(n) (S504).

The processing is branched depending on whether or not it is determined that the sudden noise correlated to the potential signal d(n) is mixed (S506). When it is determined that the noise correlated to the potential signal d(n) is mixed in the potential signal d(n), the processing is advanced to S508, and when it is not determined that the noise correlated to the potential signal d(n) is mixed in the potential signal d (n), the processing is advanced to S510.

When it is determined that the noise correlated to the potential signal d(n) is mixed, the step size setting unit 64 of the filter control unit 60e sets the step-size parameter to a first value (S508). Subsequently, the processing is advanced to S512.

When it is not determined that the noise correlated to the potential signal d(n) is mixed, the step size setting unit 64 of the filter control unit 60e sets the step-size parameter to a second value that is smaller than the first value (S510). Subsequently, the processing is advanced to S512 and is converged.

The coefficient adjusting unit 34 updates the coefficient W(n) such that an error signal H(n)×x(n)−W(n)×x (n) is minimized, based on the bioimpedance signal x(n) and the biological information signal e(n) (S512). The variable coefficient filtering unit 32 multiplies the bioimpedance signal x(n) by the coefficient W(n) to generate a control signal W(n)×x(n) that is correlated to the bioimpedance signal x(n) (S514).

The subtractor 42 of the biological information signal output unit 40 removes the control signal W(n)×x(n) predicted from the potential signal d(n) on which the motion artifact is superimposed. The biological information signal output unit 40 thereby outputs the biological information signal e(n) from which the control signal W(n)×x(n) is removed (S516). The processing is terminated by the above.

Through such processing, also with the biological information providing apparatus 200e, even when sudden noise is mixed in the measurement of the biological information signal e(n), the convergence speed of the adaptive filter 30 to remove the noise can be appropriately followed. Furthermore, the main signal e(n) can be prevented from being attenuated together with the control signal W(n)×x(n) due to an excessive coefficient updating frequency.

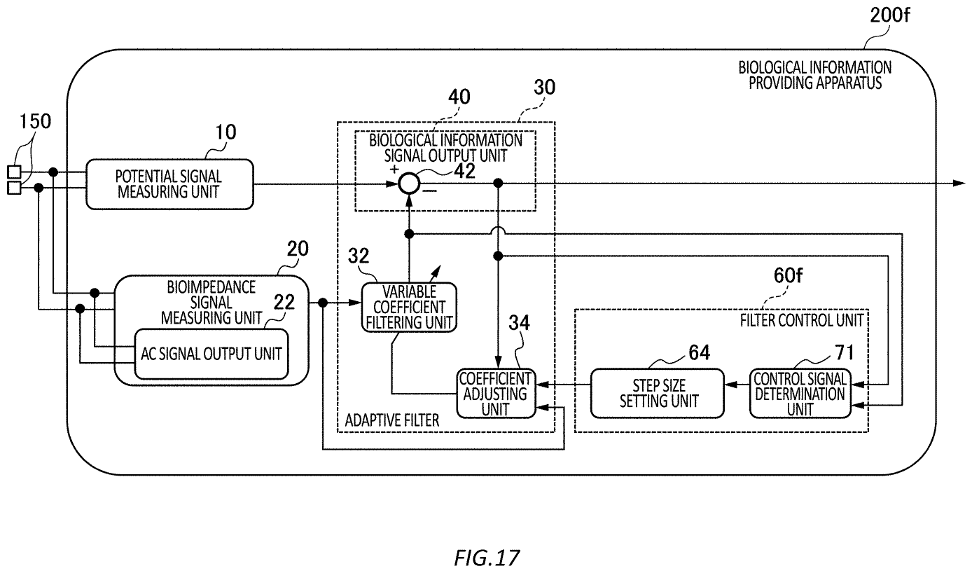

FIG. 17 illustrates an example of a block diagram of a configuration of a biological information providing apparatus 200f according to a sixth embodiment. Description will be made focusing on the difference from the biological information providing apparatus 200a according to the first embodiment. The biological information providing apparatus 200f includes a filter control unit 60f.

The filter control unit 60f determines whether or not the magnitude of the biological information signal e(n) satisfies a predetermined condition based on the control signal output by the variable coefficient filtering unit 32 and the biological information signal e(n) output by the biological information signal output unit 40, and controls the convergence speed of the adaptive filter 30 based on the determination. The filter control unit 60f includes a control signal determination unit 71 and the step size setting unit 64.

The control signal determination unit 71 determines whether the magnitude of the biological information signal e(n) satisfies the predetermined condition based on the control signal W(n)×x(n) and the biological information signal e(n). In this case, the determination may be made based on the correlation between the control signal W(n)×x(n) and the biological information signal e(n) to determine the presence or absence of mixture of the noise correlated to the MA mixed in the biological information signal e(n).

The control signal determination unit 71 is connected to the variable coefficient filtering unit 32 and the biological information signal output unit 40.

The step size setting unit 64 sets the value of the step-size parameter μ via the coefficient adjusting unit 34 based on the determination result of the control signal determination unit 71. The filter control unit 60f thereby controls the convergence speed of the adaptive filter based on the control signal W(n)×x(n) and the biological information signal e(n).

Through such processing, also with the biological information providing apparatus 200f, the convergence speed of the adaptive filter 30 to remove the noise in the measurement of the biological information signal e(n) can be appropriately followed. Furthermore, the main signal e(n) can be prevented from being attenuated together with the control signal W(n)×x(n) due to an excessive coefficient updating frequency.

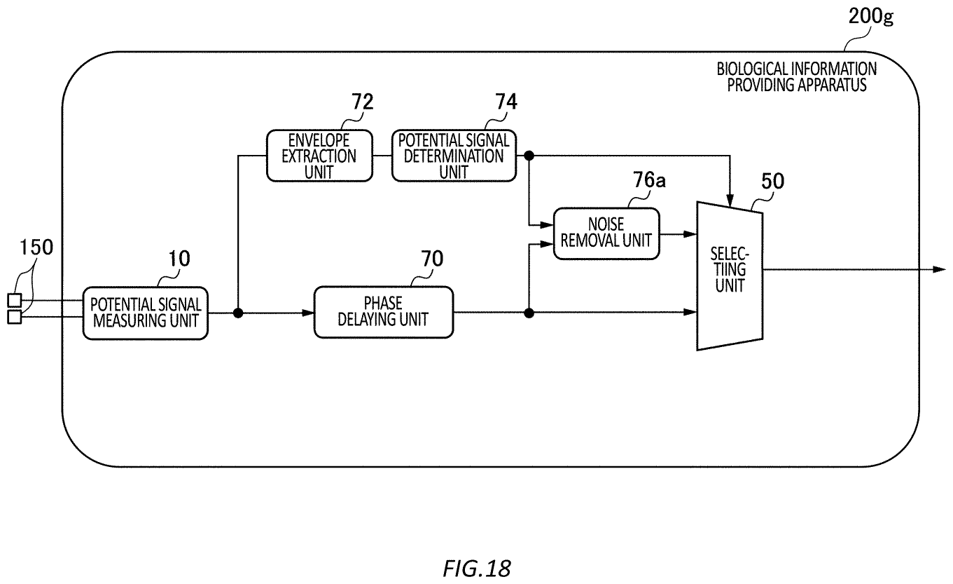

FIG. 18 illustrates an example of a block diagram of a configuration of a biological information providing apparatus 200g according to a seventh embodiment. The biological information providing apparatus 200g includes a potential signal measuring unit 10 connected to a pair of electrodes 150 in contact with a living body, a phase delaying unit 70, an envelope extraction unit 72, a potential signal determination unit 74, a noise removal unit 76a, and a selecting unit 50.

The potential signal measuring unit 10 outputs the potential signal d(n) based on the measurement result. Sudden noise may be mixed in the potential signal d(n).

The envelope extraction unit 72 performs envelope extraction on the potential signal d(n), similarly to the components included in the filter control unit 60d of the biological information providing apparatus 200d. The envelope extraction unit 72 outputs a reference waveform signal through envelope extraction including absolute value conversion and low pass filtering. The potential signal determination unit 74 can thereby easily find mixture of the sudden noise based on the reference waveform signal. The envelope extraction unit 72 may have a modulator, a mixer, and/or an arithmetic unit or the like, similarly to the envelope extraction unit 66.

The potential signal determination unit 74 determines whether or not the magnitude of the potential signal d(n) exceeds the magnitude of the reference waveform signal. The potential signal determination unit 74 may output a signal having a potential at different levels according to the determination result. The signal output by the potential signal determination unit 74 may be a pulse signal, or may have a configuration in which a signal of a predetermined level is continuously output for a predetermined period. The potential signal determination unit 74 determines that the potential signal d(n) satisfies a predetermined condition when the magnitude of the potential signal d(n) exceeds the magnitude of the reference waveform signal. As an example, depending on the signal of a different level output according to the determination result, the potential signal determination unit 74 controls which of the signals the selecting unit 50 uses as the biological information signal to be output from the biological information providing apparatus 200g.

The phase delaying unit 70 delays the phase of the potential signal d(n) to be input to the selecting unit 50 and the noise removal unit 76a by the delay time due to the envelope extraction of the envelope extraction unit 72. When the potential signal determination unit 74 determines that the sudden noise is mixed in d(n), the phase delaying unit 70 is thereby able to input a signal according to the potential signal before that time point to the selecting unit 50 and the noise removal unit 76a. Herein, the phase delaying unit 70 may delay the signal by a delay time due to the envelope extraction added with further delay time, such that, when the envelope extraction unit 72 detects the sudden noise, the noise processing performed by the noise removal unit 76a is performed according to the potential signal at a time point sufficiently preceding the time point when the sudden noise is detected.

When the potential signal determination unit 74 determines that the potential signal d(n) satisfies a predetermined condition, the noise removal unit 76a outputs a signal according to the potential signal d(n) before it is determined that the potential signal d(n) satisfies the condition.

As an example, the noise removal unit 76a may include a flip-flop. In this case, the noise removal unit 76a latches, with the flip-flop, the potential signal d(n) before it is determined that the potential signal d(n) satisfies the condition, and outputs a signal according to the potential for a predetermined time. The potential while the sudden noise is mixed is thereby held at a certain value, and the mixture of the sudden noise in the potential signal d(n) can be simply removed. As another example, the noise removal unit 76a may include a timer and a register. In this case, the potential signal d(n) before it is determined that the potential signal d(n) satisfies the condition is stored in the register, and at a predetermined time measured by the timer, a signal of the potential level stored in the register can be defined as the noise removal signal. In this manner, the noise removal unit 76a may have various components that is capable of outputting a signal according to the signal before the mixture of the sudden noise, while the sudden noise is being mixed.

The selecting unit 50 performs selection such that the signal output by the noise removal unit 76a is output as the biological information signal, when the potential signal d(n) satisfies the predetermined condition. On the other hand, when the potential signal d(n) does not satisfy the predetermined condition, the selecting unit 50 performs selection such that the potential signal d(n) is output as the biological information signal. As another example, the selecting unit 50 may perform selection such that the potential signal d(n) is replaced with a blank signal for a predetermined period, when the potential signal d(n) satisfies the predetermined condition.

As described above, the biological information providing apparatus 200g can detect whether or not the sudden noise is mixed in the potential signal d(n), and simply remove the noise when it is determined that the sudden noise is mixed in the potential signal d(n). The components at the back stage of the potential signal measuring unit 10 in the biological information providing apparatus 200g can be included in the components of the filter control units 60a to 60f of the biological information providing apparatuses 200a to 200f.

In this case, when the filter control unit detects the sudden noise indicating that the potential signal d(n) or the bioimpedance signal x(n) has a predetermined magnitude, each filter control unit 60 may perform control of the biological information providing apparatus 200g. On the other hand, when the sudden noise is not detected, each filter control unit 60 may perform control to vary the step-size parameter μ described for the biological information providing apparatuses 200a to 200f.

That is, the filter control unit 60, including the components of the biological information providing apparatus 200g, may output, as the biological information signal e(n), a signal according to the potential signal d before it is determined that the bioimpedance signal x(n) or the potential signal d(n) satisfies the condition, when the magnitude of the bioimpedance signal x(n) or the magnitude of the potential signal d(n) exceeds the predetermined first threshold value, and output, as the biological information signal e(n), the potential signal d(n), when the magnitude of the bioimpedance signal x(n) or the magnitude of the potential signal d(n) does not exceed the predetermined first threshold value (for example, 1 mV). Furthermore, the filter control unit 60 may control the coefficient adjusting unit 34 to set the step-size parameter μ of the coefficient adjusting unit 34 to a first value (for example, μ=0.5), when the magnitude of the bioimpedance signal x(n) or the magnitude of the potential signal d(n) is equal to or less than the first threshold value and exceeds a second threshold value (for example, 0.25 mV) that is lower than the first threshold value. The filter control unit 60 may control the coefficient adjusting unit 34 to set the step-size parameter μ of the coefficient adjusting unit 34 to a second value (for example, μ=0.2) that is smaller than the first value, when the magnitude of the bioimpedance signal x(n) or the magnitude of the potential signal d(n) is equal to or less than the second threshold value.

In this manner, the biological information providing apparatus 200 may perform the noise removal process on the potential signal d(n) when the sudden noise is mixed, according to the plurality of thresholds. On the other hand, when the sudden noise is not mixed, the noise removal processing may be performed by processing in which the convergence speed of the adaptive filter 30 is changed.

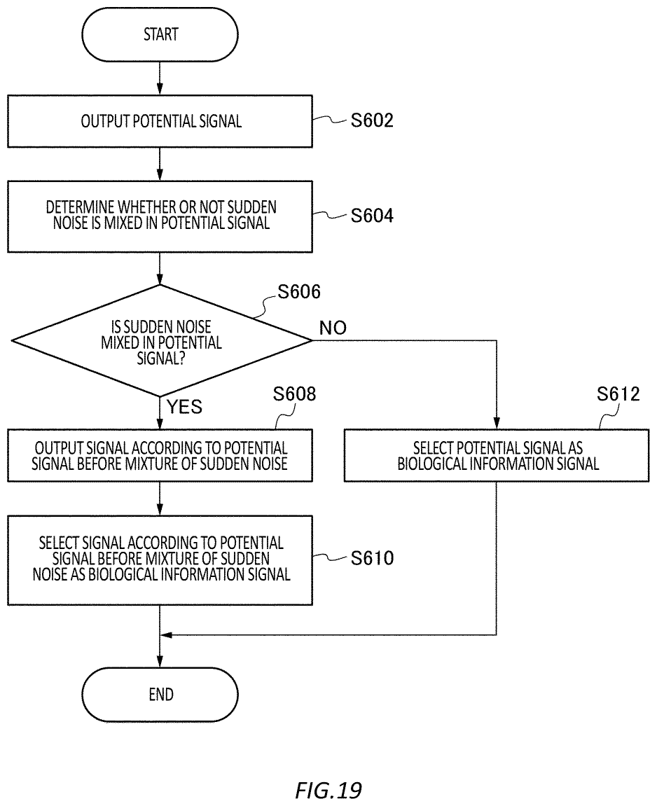

FIG. 19 is a flow diagram showing an example of processing for sudden noise removal performed at the biological information providing apparatus 200g. The processing includes steps S602 to S612.

The potential signal measuring unit 10 outputs the potential signal d(n) (S602). The potential signal determination unit 74 determines whether or not sudden noise is mixed in the potential signal d(n) (S604). The determination performed by the potential signal determination unit 74 may be performed according to the reference waveform signal obtained through the envelope extraction performed on the potential signal d(n) by the envelope extraction unit 72.

The processing is branched depending on whether or not the potential signal determination unit 74 determines that the sudden noise is mixed in the potential signal (S606). When the potential signal determination unit 74 determines that the sudden noise is mixed in the potential signal, the processing is advanced to S608. On the other hand, when the potential signal determination unit 74 does not determine that the sudden noise is mixed in the potential signal, the processing is advanced to S612.

When the potential signal determination unit 74 determines that the sudden noise is mixed in the potential signal d(n), the potential signal determination unit 74 outputs a signal that controls the selecting unit 50 and the noise removal unit 76a assuming that the sudden noise is mixed in the potential signal. The noise removal unit 76a thereby outputs a signal according to the potential signal d(n) before it is determined that the potential signal d(n) satisfies the condition, that is, before the mixture of the sudden noise (S608).

The selecting unit 50 outputs, as the biological information signal, the signal according to the potential signal d(n) before the mixture of the sudden noise, when the potential signal d(n) satisfies a predetermined condition (S610). Subsequently, the processing is terminated.

The selecting unit 50 outputs, as the biological information signal, the potential signal d(n) output by the potential signal measuring unit 10, when the potential signal d(n) does not satisfy the predetermined condition (S612). Subsequently, the processing is converged with the processing after step S610, and is terminated.

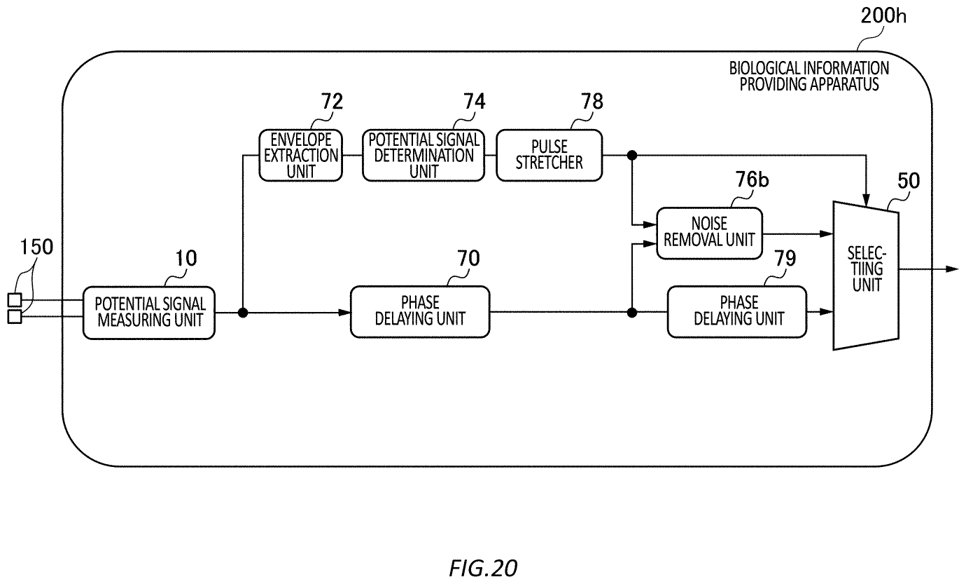

FIG. 20 illustrates an example of a block diagram of a configuration of a biological information providing apparatus 200h according to an eighth embodiment. The biological information providing apparatus 200h includes a potential signal measuring unit 10 connected to a pair of electrodes 150 in contact with a living body 500, a phase delaying unit 70, an envelope extraction unit 72, a potential signal determination unit 74, a pulse stretcher 78, a noise removal unit 76b, a phase delaying unit 79, and a selecting unit 50.

Hereinafter, the configuration of the biological information providing apparatus 200h will be described focusing on the difference from the biological information providing apparatus 200g. The biological information providing apparatus 200h is different in the components at the back stage of the potential signal determination unit 74.

The pulse stretcher 78 outputs a signal obtained by stretching the pulse output by the potential signal determination unit 74. The pulse stretcher 78 can thereby enable the selecting unit 50 to select signals, from the noise removal unit 76b, on which noise removal has been performed for a sufficiently long period before and after a period equivalent to the period during which the sudden noise was mixed in the potential signal d(n).

The phase delaying unit 70 delays the phase of the potential signal d(n) to be input to the selecting unit 50 and the noise removal unit 76a by the delay time due to the envelope extraction of the envelope extraction unit 72. The phase delaying unit 70 is equivalent to the “first phase delaying unit”.

When the potential signal determination unit 74 determines that the potential signal d(n) satisfies a predetermined condition, the noise removal unit 76b outputs a signal according to the potential signal d(n) before it is determined that the potential signal d(n) satisfies the condition. Herein, when the noise is a sudden one, the frequency of the sudden noise becomes higher. Therefore, the noise removal unit 76b can remove the noise mixed in the potential signal d(n) by performing low pass filtering. Specifically, the noise removal unit 76b performs the low pass filtering on the output signal of the phase delaying unit 70, and outputs a signal according to the potential signal d(n) before it is determined that the potential signal d(n) satisfies the predetermined condition.

The phase delaying unit 79 delays the phase of the potential signal d(n) to be input to the selecting unit 50 by a delay time due to the low pass filtering by the noise removal unit 76b. The phase delaying unit 79 is equivalent to the “second phase delaying unit”.

The selecting unit 50 performs selection such that the signal output by the noise removal unit 76a is output as the biological information signal, when the potential signal d(n) satisfies the predetermined condition. On the other hand, when the potential signal d(n) does not satisfy the predetermined condition, the selecting unit 50 performs selection such that the potential signal d(n) is output as the biological information signal. As already described, in the present embodiment, the period during which the potential signal d(n) satisfies the predetermined condition may be set by the pulse stretcher 78 as a period that is longer before and after said period. Therefore, the selecting unit 50 in the present embodiment can ensure the noise removal for the period before and after when the sudden noise may be mixed. Note that, the selecting unit 50 may select a blank signal as the biopotential signal when the potential signal d(n) satisfies the predetermined condition.

As described above, the biological information providing apparatus 200h detects whether or not the sudden noise is mixed in the potential signal d(n). The biological information providing apparatus 200h can remove the noise with a component other than the biological information providing apparatus 200g, when it is determined that the sudden noise is mixed in the potential signal d(n).

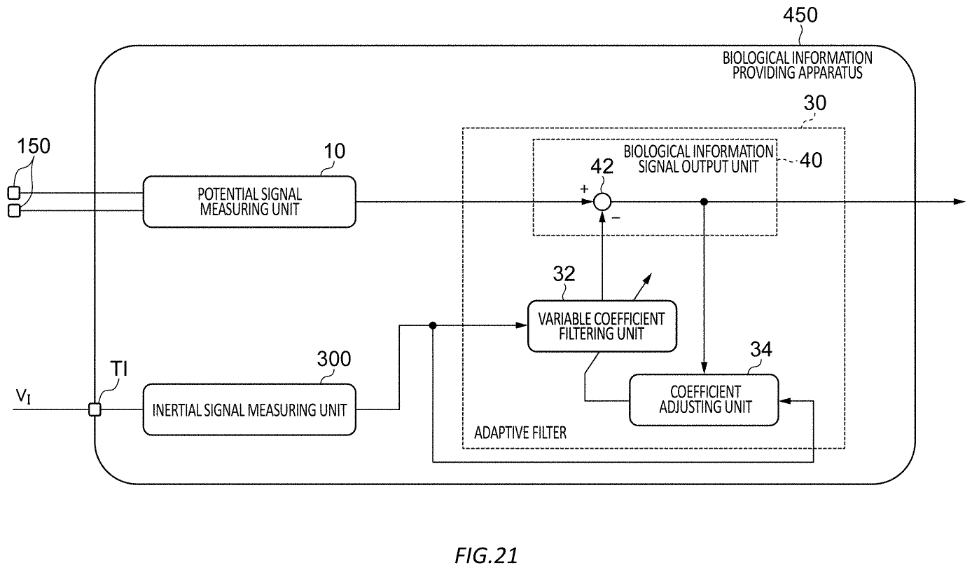

FIG. 21 illustrates an example of a block diagram of a configuration of a biological information providing apparatus 450 according to a comparative example. The biological information providing apparatus 450 includes a potential signal measuring unit 10 connected to a pair of electrodes 150 in contact with a living body 500, an adaptive filter 30, and an inertial signal measuring unit 300. Unlike the biological information providing apparatus 100 according to FIG. 2 or the like or the biological information providing apparatus 200 according to FIG. 5 or the like, the biological information providing apparatus 450 does not have an AC signal output unit 22 included in the bioimpedance signal measuring unit 20 and the bioimpedance signal measuring unit 20.

The inertial signal measuring unit 300 measures an acceleration or angular velocity of the living body 500 to detect an inertial motion of the living body 500, and outputs an inertial signal. In the measurement of the potential signal, the noise based on the motion artifact is noise generated based on a motion of the living body 500.

In an embodiment where the biological information providing apparatus 200 includes the bioimpedance signal measuring unit 20, the biological information providing apparatus 200 detected the noise generated based on the motion of the living body 500 as a variation in the bioimpedance. In the ninth embodiment, the inertial signal measuring unit 300 measures such noise in the potential signal associated with the motion of the living body 500 by measuring the acceleration or angular velocity associated with the motion of the living body 500.

For example, the inertial signal measuring unit 300 is connected to an inertial measuring unit (IMU) via an external connection terminal TI provided in the biological information providing apparatus 450. Note that, a sensor connected to the terminal TI as the sensor for providing the inertial signal may be an acceleration sensor and/or a gyro sensor, instead of the IMU. Therefore, a signal V_{I} from the inertial measuring unit (IMU) is applied to the terminal TI of the biological information providing apparatus 450 to which the inertial signal measuring unit 300 is connected. As an example, the signal V_{I} is a voltage signal. In this manner, the IMU may be provided outside the biological information providing apparatus 450, or may be provided in the biological information providing apparatus 450.

As such, in the comparative example, the biological information providing apparatus 450 is capable of detecting the noise associated to a movement of the living body 500 included in the potential signal, when it does not directly measure the bioimpedance signal. Therefore, the biological information providing apparatus 450 is capable of reducing the noise included in the potential signal even when the living body 500 moves.

The adaptive filter 30 is a filter for removing the noise due to a variation in the inertial signal from a potential signal V_{EMG} caused by a composite action potential measured by the potential signal measuring unit 10. The adaptive filter 30 includes a variable coefficient filtering unit 32, a coefficient adjusting unit 34, and a biological information signal output unit 40. Description of the adaptive filter 30 will be omitted since it has already been described for other embodiments.

The variable coefficient filtering unit 32 of the biological information providing apparatus 450 according to the comparative example generates a control signal based on an inertial signal and a coefficient. The variable coefficient filtering unit 32 may generate a control signal y(n) by an approach similar the output of the control signal y(n) performed in the comparative example of FIG. 3. Note that, in the case of the ninth embodiment, the bioimpedance signal measuring unit in FIG. 3 is replaced with the biological information providing apparatus 450. The coefficient adjusting unit 34 generates a control signal y(n)=W(n)×x(n) based on the inertial signal and the coefficient W(n). The biological information signal output unit 40 thereby calculates a biological information signal e(n)=d(n)−y(n) based on the potential signal d(n) and the control signal y(n), and outputs the biological information signal e(n).

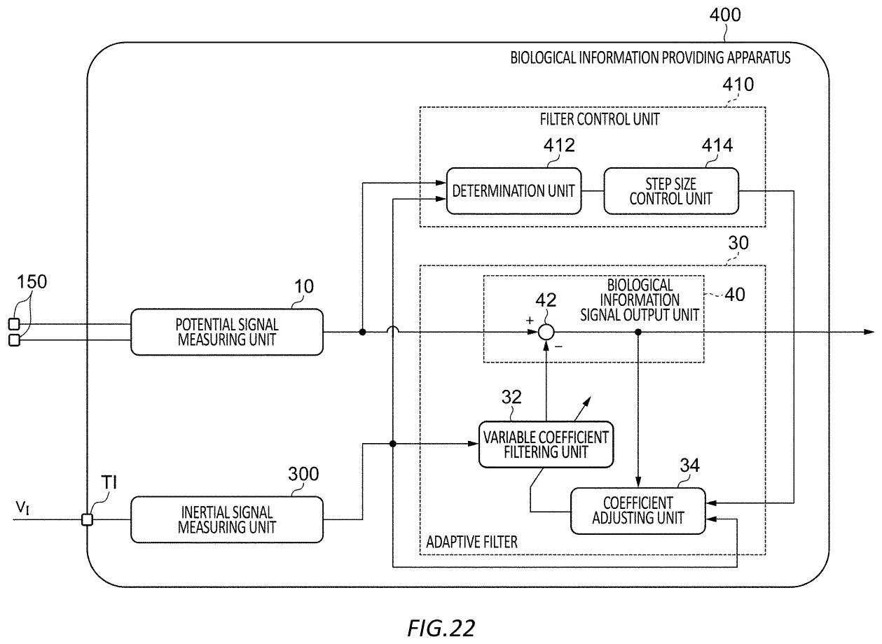

FIG. 22 illustrates an example of a block diagram of a configuration of a biological information providing apparatus 400 according to a ninth embodiment. The biological information providing apparatus 400 according to the ninth embodiment includes a filter control unit 410 in addition to the components of the biological information providing apparatus 450 according to the comparative example. Hereinafter, the ninth embodiment will be described focusing on the difference from the comparative example.

The filter control unit 410 controls the coefficient adjusting unit 34 such that the convergence speed of the adaptive filter 30 is faster in a case where the potential signal or the inertial signal satisfies the predetermined condition than in a case where the potential signal or the inertial signal does not satisfy the condition. The filter control unit 410 includes a determination unit 412 and a step size control unit 414.

The determination unit 412 determines whether or not the potential signal or the inertial signal satisfies the predetermined condition. Thus, both the potential signal of the output by the potential signal measuring unit 10 and the inertial signal output by the inertial signal measuring unit 300 are input to the determination unit 412 of the present embodiment.

Similar to other embodiments already described, when the determination unit 412 determines that the potential signal or the inertial signal satisfies the predetermined condition, the motion of the living body 500 becomes intensive and the noise caused by the motion artifact in the potential signal measured by the pair of electrodes 150 in contact with the living body 500 increases. That is, in this case, sudden noise caused by the motion artifact is generated in the measured potential signal. The determination unit 412 determines whether or not the sudden noise is generated in the potential signal or the inertial signal correlated to the noise in the potential signal associated with the motion of the living body 500.

The determination unit 412 may determine that the inertial signal or the potential signal satisfies the predetermined condition, when the magnitude of the inertial signal or the potential signal exceeds a predetermined threshold (for example, 1 mV). This predetermined threshold is an example of the “first threshold value”. Note that, more thresholds may be set than in the present embodiment, and accordingly, the step-size parameter μ may be switched in even more steps than in the present embodiment.

Note that, the filter control unit 410 may include, at a front stage of the determination unit 412, an envelope extraction unit 66 included in the biological information providing apparatus 200b and the biological information providing apparatus 200d, or an envelope extraction unit (not shown) such as the envelope extraction unit 72 included in the biological information providing apparatus 200g. In this case, the determination unit 412 may determine that the inertial signal satisfies the condition, when the magnitude of the inertial signal or the potential signal exceeds the magnitude of the reference waveform signal obtained by performing envelope extraction on the inertial signal.

In this case, the biological information providing apparatus 400 may include a phase delaying unit 70 (not shown) at a front stage of the adaptive filter 30, similarly to the biological information providing apparatus 200b and the biological information providing apparatus 200d. The phase delaying unit delays the phase of the potential signal input to the biological information signal output unit 40 by a delay time due to the envelope extraction performed by the envelope extraction unit 66 or the envelope extraction unit 72.

The determination unit 412 may include a selector, for example. While the inertial signal and the potential signal are input to the determination unit 412, the determination unit 412 selects the input signal by the selector. The determination unit 412 functions as the biological information providing apparatus 400 of the tenth embodiment described below or the eleventh embodiment described below by comparing the selected signal with the predetermined threshold.

Furthermore, the determination unit 412 may determine that the potential signal or the inertial signal satisfies the predetermined condition, when the degree of correlation between the potential signal output by the potential signal measuring unit 10 and the inertial signal output by the inertial signal measuring unit 300 exceeds a predetermined threshold. In this case, the determination unit 412 performs comparison between the potential signal and the inertial signal input to the determination unit 412 via the selector.

The step size control unit 414 may set the step-size parameter to a first value (for example, μ=0.5), when the determination unit 412 determines that the potential signal or the inertial signal satisfies the predetermined condition. The step size control unit 414 may set the step-size parameter to a second value(for example, μ=0.2) that is smaller than the first value, when the determination unit 412 determines that the potential signal or the inertial signal does not satisfy the predetermined condition. The filter control unit 410 may thereby control the adaptive filter 30 such that the convergence speed of the adaptive filter 30 is faster.

In this manner, the step size control unit 414 increases the step-size parameter μ to sufficiently remove the influence of the noise from the biological information signal, when the sudden noise is generated in the potential signal or the inertial signal correlated to the noise in the potential signal associated with the motion of the living body 500. On the other hand, the step size control unit 414 reduces the step-size parameter μ to prevent the coefficient adjusting unit 34 from outputting a control signal that attenuates the main signal, when the sudden noise is not generated in the potential signal or the inertial signal.

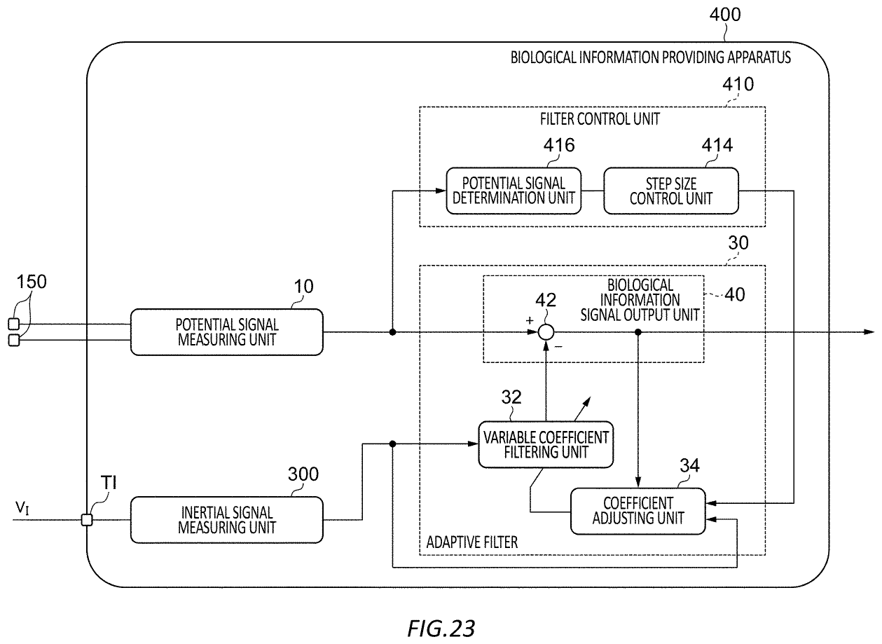

FIG. 23 illustrates an example of a block diagram of a configuration of a biological information providing apparatus 400 according to a tenth embodiment. The eleventh embodiment is equivalent to an embodiment in the ninth embodiment where, when the determination unit 412 includes a selector, the potential signal is selected by the selector to compare the magnitude of the potential signal with a predetermined threshold (for example, 1 mV).

The filter control unit 410 includes a step size control unit 414 and a potential signal determination unit 416. The potential signal determination unit 416 may determine that the potential signal satisfies a predetermined condition, when the magnitude of the potential signal exceeds a predetermined threshold. In this case, the determination unit 412 in the ninth embodiment functions as the potential signal determination unit 416 in the tenth embodiment.

The step size control unit 414 may set the step-size parameter to a first value (for example, μ=0.5), when the potential signal determination unit 416 determines that the potential signal satisfies the predetermined condition. The step size control unit 414 may set the step-size parameter to a second value(for example, μ=0.2) that is smaller than the first value, when the potential signal determination unit 416 determines that the potential signal does not satisfy the predetermined condition.

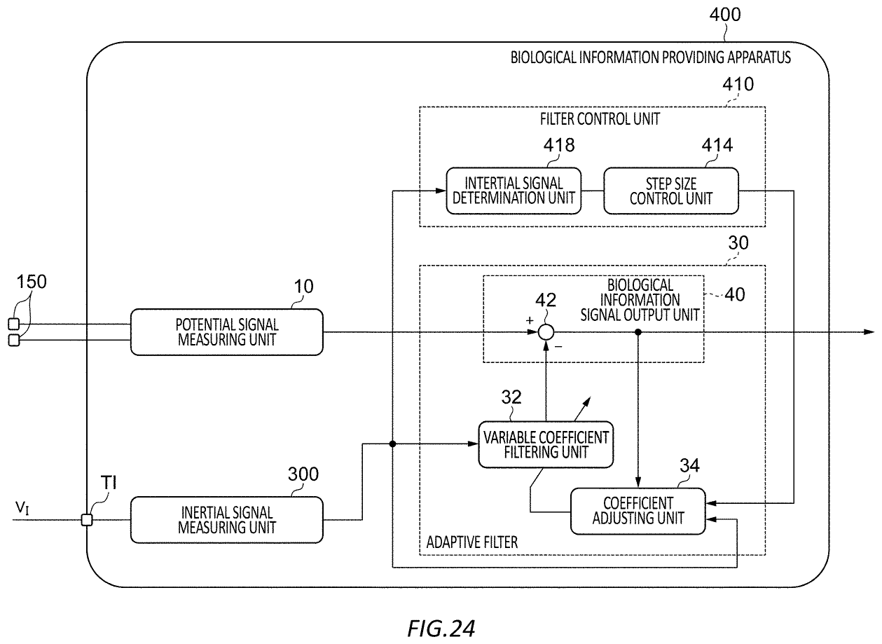

FIG. 24 illustrates an example of a block diagram of a configuration of a biological information providing apparatus 400 according to an eleventh embodiment. The twelfth embodiment is equivalent to an embodiment in the ninth embodiment where, when the determination unit 412 includes a selector, the inertial signal is selected by the selector to compare the magnitude of the inertial signal with a predetermined threshold (for example, 1 mV).

The filter control unit 410 includes a step size control unit 414 and an inertial signal determination unit 418. The inertial signal determination unit 418 may determine that the inertial signal satisfies the predetermined condition, when the magnitude of the inertial signal exceeds a predetermined threshold. In this case, the determination unit 412 in the ninth embodiment functions as the inertial signal determination unit 418 in the eleventh embodiment.

The step size control unit 414 may set the step-size parameter to a first value (for example, μ=0.5), when the inertial signal determination unit 418 determines that the inertial signal satisfies the predetermined condition. The step size control unit 414 may set the step-size parameter to a second value(for example, μ=0.2) that is smaller than the first value, when the inertial signal determination unit 418 determines that the inertial signal does not satisfy the predetermined condition.

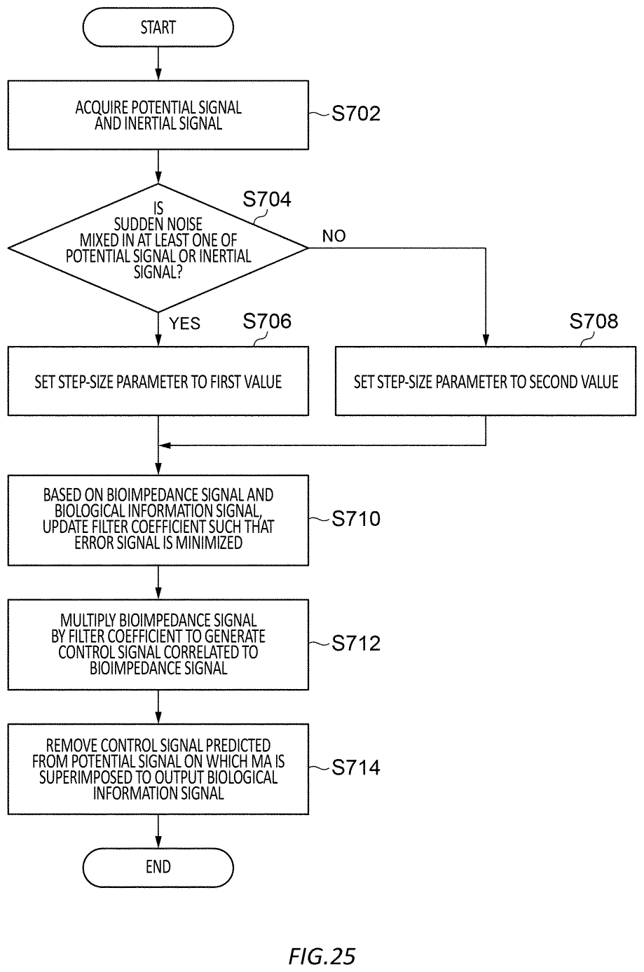

FIG. 25 is an example of a flow diagram showing the processing of the biological information providing apparatus 400 according to the ninth embodiment. The processing of the biological information providing apparatus 400 in the present embodiment includes steps S702 to S714.

The determination unit 412 acquires a potential signal output by the potential signal measuring unit 10 and an inertial signal output by the inertial signal measuring unit 300 (S702). The determination unit 412 determines whether or not sudden noise is mixed in at least one of the potential signal or the inertial signal (S704).

The determination unit 412 may select the potential signal by the selector, when the magnitude of the potential signal is to be compared with a predetermined threshold. The determination unit 412 may select the inertial signal by the selector, when the magnitude of the inertial signal is to be compared with a predetermined threshold. Alternatively, the determination unit 412 may perform comparison between the potential signal and the inertial signal input to the determination unit 412 without using the selector, and determine whether the degree of correlation between the potential signal output by the potential signal measuring unit 10 and the inertial signal output by the inertial signal measuring unit 300 exceeds a predetermined threshold.

The determination unit 412 may thereby determine whether or not the sudden noise is mixed in at least one of the potential signal or the inertial signal. When the determination unit 412 determines that the sudden noise is mixed in at least one of the potential signal or the inertial signal, the step size control unit 414 sets the step-size parameter to a first value (S706). When the determination unit 412 determines that the sudden noise is not mixed in at least one of the potential signal or the inertial signal, the step-size parameter is set to a second value (S708).

The coefficient adjusting unit 34 updates a filter coefficient such that an error signal is minimized, based on the inertial signal and the biological information signal (S710). The variable coefficient filtering unit 32 multiplies the inertial signal by the filter coefficient to generate a control signal correlated to the inertial signal (S712).

The subtractor 42 of the biological information signal output unit 40 removes the control signal predicted from the potential signal on which the motion artifact is superimposed to output a biological information signal. The biological information signal output unit 40 thereby outputs the biological information signal from which the control signal is removed (S714). The processing is terminated by the above.

Through such processing, even when sudden noise is mixed in the measurement of the biological information signal e(n), the biological information providing apparatus 400 is allowed to appropriately follow the convergence speed of the adaptive filter 30 to remove the noise. Furthermore, the biological information providing apparatus 400 can prevent the main signal e(n) from being attenuated together with the control signal due to an excessive coefficient updating frequency.

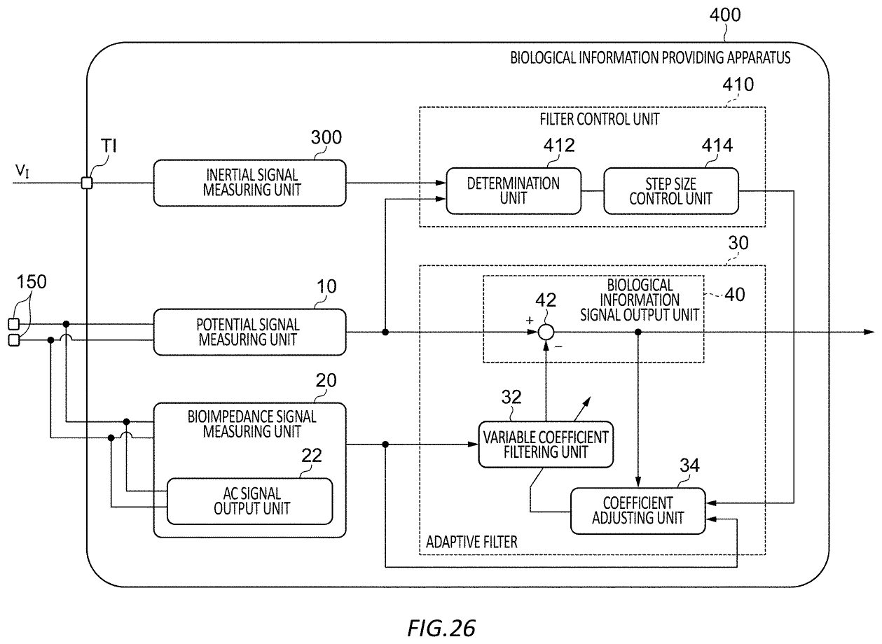

FIG. 26 illustrates an example of a block diagram of a configuration of a biological information providing apparatus 400 according to a twelfth embodiment. The biological information providing apparatus 400 includes a potential signal measuring unit 10, a bioimpedance signal measuring unit 20, an adaptive filter 30, an inertial signal measuring unit 300, and a filter control unit 410. The biological information providing apparatus 400 according to the twelfth embodiment includes both the bioimpedance signal measuring unit 20 and the inertial signal measuring unit 300.

In such regard, the biological information providing apparatus 400 according to the twelfth embodiment differs from the biological information providing apparatus 400 according to the ninth embodiment to the eleventh embodiment. The potential signal measuring unit 10 measures a potential signal through a pair of electrodes 150 in contact with a living body 500.

The bioimpedance signal measuring unit 20 measures the bioimpedance generated between the pair of electrodes 150 to output a bioimpedance signal according to the bioimpedance BioZ. In addition, the bioimpedance signal measuring unit 20 includes an AC signal output unit 22 for the potential signal measuring unit 10 to measure the potential generated in the living body and for the bioimpedance signal measuring unit 20 to measure the impedance of the living body.

The inertial signal measuring unit 300 outputs inertial signal according to an acceleration or angular velocity. The inertial signal measuring unit 300 thereby outputs the inertial signal for the determination unit 412 to determine whether or not the living body has performed a sudden movement.

The adaptive filter 30 is a filter that removes, from the potential signal V_{EMG} generated by the composite action potential measured by the potential signal measuring unit 10, noise caused by the variation in the bioimpedance signal. The adaptive filter 30 is configured to include a variable coefficient filtering unit 32, a coefficient adjusting unit 34, and a biological information signal output unit 40. Description for details of the operation of individual components of the adaptive filter 30 will be omitted since it has already been described for other embodiments.

The filter control unit 410 controls the coefficient adjusting unit such that the convergence speed of the adaptive filter 30 is faster in a case where the potential signal or the inertial signal satisfies the predetermined condition than in a case where the potential signal or the inertial signal does not satisfy the condition. The filter control unit 410 includes a determination unit 412 and a step size control unit 414. Description for the filter control unit 410 will be omitted since it is operated similarly to that in the embodiment of FIG. 22.

The operation of a combination of the adaptive filter 30 and the filter control unit 410 is different in the present embodiment from other embodiments. This difference will be described. In the present embodiment, the determination unit 412 determines mixture of sudden noise in the potential signal and the inertial signal correlated to the noise of the potential signal, based on the inertial signal or the potential signal. Thus, both the potential signal and the inertial signal are input to the determination unit 412. The filter control unit 410 of the twelfth embodiment thereby determines whether or not the noise is mixed through an operation similar to that of the filter control unit 410 of the ninth embodiment.

On the other hand, in the twelfth embodiment, unlike the biological information providing apparatus 400 in the ninth embodiment to the eleventh embodiment, the bioimpedance signal measuring unit 20 is included. The adaptive filter 30 removes, from the biopotential signal, the noise component such as the motion artifact proportional a variation in the bioimpedance, based on the bioimpedance signal output from the bioimpedance signal measuring unit 20.

In this manner, in the twelfth embodiment, the biological information providing apparatus 400 detects the sudden noise mixed in the potential signal, the inertial signal, or the bioimpedance signal, based on the inertial signal or the potential signal, and outputs the biological information signal based on the potential signal and the bioimpedance signal. The biological information providing apparatus 400 is thereby capable of accurately detecting the presence or absence of the sudden noise, and can effectively remove the influence of the noise such as the variation in the bioimpedance in the measurement of the biological information signal.

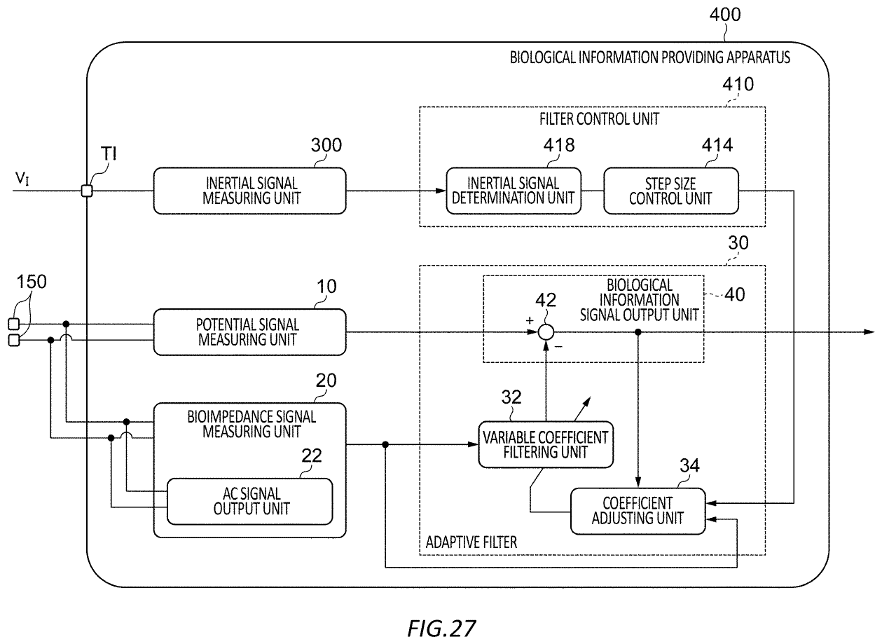

FIG. 27 illustrates an example of a block diagram of a configuration of a biological information providing apparatus 400 according to a thirteenth embodiment. The thirteenth embodiment is equivalent to an embodiment where, when the determination unit 412 includes a selector, the determination unit 412 in the twelfth embodiment selects the inertial signal by the selector to compare the magnitude of the inertial signal with a predetermined threshold (for example, 1 mV).

The filter control unit 410 includes a step size control unit 414 and an inertial signal determination unit 418. The inertial signal determination unit 418 may determine that the inertial signal satisfies the condition, when the magnitude of the inertial signal exceeds a predetermined threshold. In this case, the determination unit 412 in the twelfth embodiment functions as the inertial signal determination unit 418 in the thirteenth embodiment.

The step size control unit 414 may set the step-size parameter to a first value (for example, μ=0.5), when the inertial signal determination unit 418 determines that the inertial signal satisfies the predetermined condition. The step size control unit 414 may set the step-size parameter to a second value(for example, μ=0.2) that is smaller than the first value, when the inertial signal determination unit 418 determines that the inertial signal does not satisfy the predetermined condition.

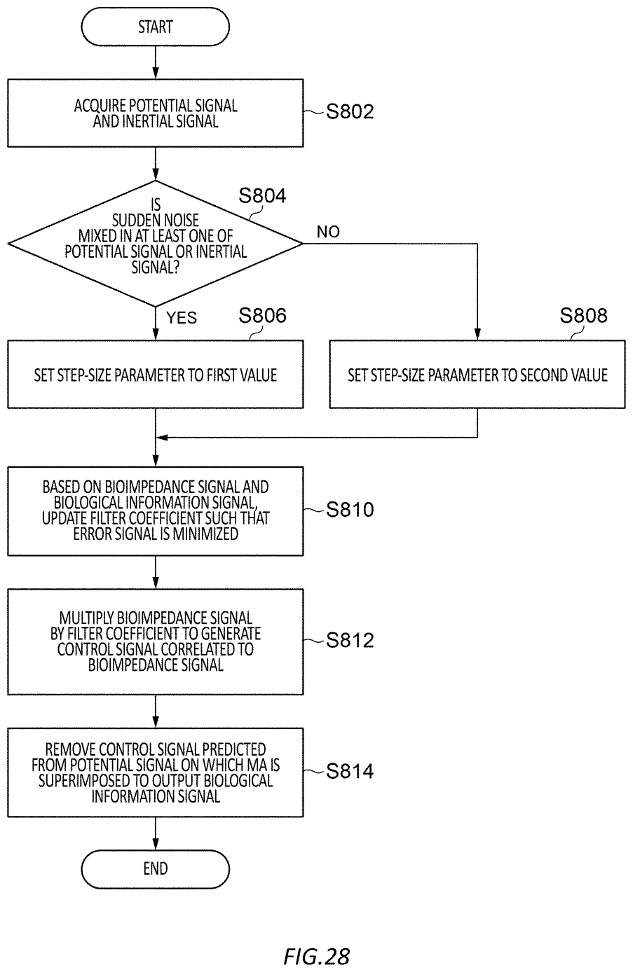

FIG. 28 is an example of a flow diagram showing processing of the biological information providing apparatus 400 according to the twelfth embodiment or the thirteenth embodiment. The processing of the biological information providing apparatus 400 in the present embodiment includes steps S802 to S814.

The determination unit 412 acquires a potential signal output by the potential signal measuring unit 10 and an inertial signal output by the inertial signal measuring unit 300 (S802). The determination unit 412 determines whether or not sudden noise is mixed in at least one of the potential signal or the inertial signal (S804).

The determination unit 412 may select the potential signal by the selector, when the magnitude of the potential signal is to be compared with a predetermined threshold. The determination unit 412 may select the inertial signal by the selector, when the magnitude of the inertial signal is to be compared with a predetermined threshold. Alternatively, the determination unit 412 may perform comparison between the potential signal and the inertial signal input to the determination unit 412 without using the selector, and determine whether the degree of correlation between the potential signal output by the potential signal measuring unit 10 and the inertial signal output by the inertial signal measuring unit 300 exceeds a predetermined threshold.

The determination unit 412 may thereby determine whether or not the sudden noise is mixed in at least one of the potential signal or the inertial signal. When the determination unit 412 determines that the sudden noise is mixed in at least one of the potential signal or the inertial signal, the step size control unit 414 sets the step-size parameter to a first value (S806). When the determination unit 412 determines that the sudden noise is not mixed in at least one of the potential signal or the inertial signal, the step-size parameter is set to a second value (S808). Note that, more thresholds may be set than in the present embodiment, and accordingly, the step-size parameter μ may be switched in even more steps than in the present embodiment.

The coefficient adjusting unit 34 updates a filter coefficient such that an error signal is minimized, based on the bioimpedance signal and the biological information signal (5810). The variable coefficient filtering unit 32 multiplies the bioimpedance signal by the filter coefficient to generate a control signal correlated to the bioimpedance signal (5812).

Also in the ninth embodiment to the twelfth embodiment, the adaptive filter 30 may adopt NLMS as the adaptation algorithm. That is, the adaptive filter 30 may be an NLMS adaptive filter.

In this case the filter control unit 410 may perform control of the stability constant ε, together with the step-size parameter μ, or alternatively instead of μ. Therefore, the filter control unit 410 may control the coefficient adjusting unit 34 to set the stability constant ε of the adaptive filter 30 to a predetermined first value, when the potential signal or the inertial signal satisfies the predetermined condition. On the other hand, the filter control unit 410 may control the coefficient adjusting unit 34 to set the stability constant ε of the adaptive filter 30 to a predetermined second value that is greater than the first value, when the potential signal or the inertial signal does not satisfy the predetermined condition.

In another embodiment, the filter control unit 410 may control the coefficient adjusting unit 34 such that the operation processing speed of the adaptive filter 30 is at a first operation processing speed, when the potential signal or the inertial signal satisfies the predetermined condition. On the other hand, the filter control unit 410 may control the coefficient adjusting unit 34 such that the operation processing speed of the adaptive filter 30 is at a second operation processing speed that is slower than the first operation processing speed, when the potential signal or the inertial signal does not satisfy the condition.

Further, in another embodiment, the filter control unit 410 may control the coefficient adjusting unit 34 such that an update interval of the coefficient is at a first interval, when the potential signal or the inertial signal satisfies the predetermined condition. On the other hand, the filter control unit 410 may control the coefficient adjusting unit 34 such that the update interval of the coefficient is at a second interval that is longer than the first interval, when the potential signal or the inertial signal does not satisfy the condition.

The subtractor 42 of the biological information signal output unit 40 removes the control signal predicted from the potential signal on which the motion artifact is superimposed to output a biological information signal. The biological information signal output unit 40 thereby outputs the biological information signal from which the control signal is removed (5814). The processing is terminated by the above.

Through such processing, also with the biological information providing apparatus 400, even when sudden noise is mixed in the measurement of the biological information signal, the biological information providing apparatus 400 is allowed to appropriately follow the convergence speed of the adaptive filter 30 to remove the noise. Furthermore, the biological information providing apparatus 400 can prevent the main signal e(n) from being attenuated together with the control signal due to an excessive coefficient updating frequency. [0267] While the present invention has been described above by way of the embodiments, the technical scope of the present invention is not limited to the scope described in the above-described embodiments. It is apparent to persons skilled in the art that various alterations or improvements can be made to the above-described embodiments. It is also apparent from the description of the claims that the form to which such alterations or improvements are made can be included in the technical scope of the present invention.

It should be noted that the operations, procedures, steps, stages, and the like of each process performed by an apparatus, system, program, and method shown in the claims, the specification, or the drawings can be realized in any order as long as the order is not indicated by “prior to,” “before,” or the like and as long as the output from a previous process is not used in a later process. Even if the operation flow is described by using phrases such as “first” or “next” for the sake of convenience in the claims, specification, and drawings, it does not necessarily mean that the process must be performed in this order.

### EXPLANATION OF REFERENCES

10: potential signal measuring unit,
12: noise mixture portion,
14: adder
20: bioimpedance signal measuring unit,
22: AC signal output unit,
30: adaptive filter,
32: variable coefficient filtering unit,
34: coefficient adjusting unit,
40: biological information signal output unit,
42: subtractor,
50: selecting unit,
60: filter control unit,
62: bioimpedance signal determination unit,
64: step size setting unit,
66: envelope extraction unit,
67: potential signal determination unit,
69: correlation determination unit,
70, 79: phase delaying unit,
72: envelope extraction unit,
74: potential signal determination unit,
76: noise removal unit,
78: pulse stretcher,
82, 84, 91, 93, 95, 99: polygonal line,
97: frame,
100, 200: biological information providing apparatus,
150: electrode,
300: inertial signal measuring unit,
400: biological information providing apparatus,
410: filter control unit,
412: determination unit,
414: step size control unit,
416: potential signal determination unit,
418: inertial signal determination unit,
450: biological information providing apparatus,
500: living body,
502: epidermal layer,
504: dermal and subcutaneous layers,
506: muscle layer,
508: muscle fiber,
510: nerve.

What is claimed is:

1. A biological information providing apparatus comprising:
a potential signal measuring unit which measures a potential signal through a pair of electrodes in contact with a living body;
a bioimpedance signal measuring unit which measures a bioimpedance that occurs between the pair of electrodes and outputs a bioimpedance signal according to the bioimpedance;
an adaptive filter having a variable coefficient filtering unit which generates a control signal based on the bioimpedance signal and a coefficient, a biological information signal output unit which calculates a biological information signal based on the potential signal and the control signal, and a coefficient adjusting unit which adjusts the coefficient such that a signal correlated to the bioimpedance signal included in the biological information signal is reduced; and
a filter control unit which controls the coefficient adjusting unit such that a convergence speed of the adaptive filter is faster in a case where the potential signal or the bioimpedance signal satisfies a predetermined condition than in a case where the potential signal or the bioimpedance signal does not satisfy the condition.

2. The biological information providing apparatus according to claim 1, wherein the filter control unit determines that the bioimpedance signal satisfies the condition when a magnitude of the bioimpedance signal exceeds a predetermined first threshold value.

3. The biological information providing apparatus according to claim 1, wherein the filter control unit determines that the bioimpedance signal satisfies the condition when a magnitude of the bioimpedance signal exceeds a magnitude of a reference waveform signal obtained by performing envelope extraction on the bioimpedance signal.

4. The biological information providing apparatus according to claim 3, further comprising a phase delaying unit which delays a phase of the potential signal input to the biological information signal output unit by a delay time due to the envelope extraction.

5. The biological information providing apparatus according to claim 1, wherein the filter control unit determines that the potential signal satisfies the condition when a magnitude of the potential signal exceeds a predetermined first threshold value.

6. The biological information providing apparatus according to claim 1, wherein the filter control unit determines that the potential signal satisfies the condition when a magnitude of the potential signal exceeds a magnitude of a reference waveform signal obtained by performing envelope extraction on the potential signal.

7. The biological information providing apparatus according to claim 6, further comprising a phase delaying unit which delays a phase of the potential signal input to the biological information signal output unit by a delay time due to the envelope extraction.

8. The biological information providing apparatus according to claim 1, wherein the filter control unit determines that the potential signal or the bioimpedance signal satisfies the condition when a degree of correlation between the potential signal and the bioimpedance signal exceeds a predetermined threshold.

9. A biological information providing apparatus comprising:
a potential signal measuring unit which measures a potential signal through a pair of electrodes in contact with a living body;
an inertial signal measuring unit which outputs an inertial signal according to acceleration or angular velocity;
an adaptive filter having a variable coefficient filtering unit which generates a control signal based on the inertial signal and a coefficient, a biological information signal output unit which calculates a biological information signal based on the potential signal and the control signal, and a coefficient adjusting unit which adjusts the coefficient such that a signal correlated to the inertial signal included in the biological information signal is reduced; and
a filter control unit which controls the coefficient adjusting unit such that a convergence speed of the adaptive filter is faster in a case where the potential signal or the inertial signal satisfies a predetermined condition than in a case where the potential signal or the inertial signal does not satisfy the condition.

10. The biological information providing apparatus according to claim 9, wherein the filter control unit determines that the inertial signal satisfies the condition when a magnitude of the inertial signal exceeds a predetermined first threshold value.

11. The biological information providing apparatus according to claim 9, wherein the filter control unit determines that the inertial signal satisfies the condition when a magnitude of the inertial signal exceeds a magnitude of a reference waveform signal obtained by performing envelope extraction on the inertial signal.

12. The biological information providing apparatus according to claim 11, further comprising a phase delaying unit which delays a phase of the potential signal input to the biological information signal output unit by a delay time due to the envelope extraction.

13. The biological information providing apparatus according to claim 9, wherein the filter control unit determines that the potential signal satisfies the condition when a magnitude of the potential signal exceeds a predetermined first threshold value.

14. The biological information providing apparatus according to claim 9, wherein the filter control unit determines that the potential signal satisfies the condition when a magnitude of the potential signal exceeds a magnitude of a reference waveform signal obtained by performing envelope extraction on the potential signal.

15. The biological information providing apparatus according to claim 14, further comprising a phase delaying unit which delays a phase of the potential signal input to the biological information signal output unit by a delay time due to the envelope extraction.

16. The biological information providing apparatus according to claim 9, wherein the filter control unit determines that the potential signal or the inertial signal satisfies the condition when a degree of correlation between the potential signal and the inertial signal exceeds a predetermined threshold.

17. A biological information providing apparatus comprising:
a potential signal measuring unit which measures a potential signal through a pair of electrodes in contact with a living body;
a bioimpedance signal measuring unit which measures a bioimpedance that occurs between the pair of electrodes and outputs a bioimpedance signal according to the bioimpedance;
an inertial signal measuring unit which outputs an inertial signal according to acceleration or angular velocity;
an adaptive filter having a variable coefficient filtering unit which generates a control signal based on the bioimpedance signal and a coefficient, a biological information signal output unit which calculates a biological information signal based on the potential signal and the control signal, and a coefficient adjusting unit which adjusts the coefficient such that a signal correlated to the bioimpedance signal included in the biological information signal is reduced; and
a filter control unit which controls the coefficient adjusting unit such that a convergence speed of the adaptive filter is faster in a case where the inertial signal satisfies a predetermined condition than in a case where the inertial signal does not satisfy the condition.

18. The biological information providing apparatus according to any one of claims 1 to 8, wherein
the filter control unit controls the coefficient adjusting unit to set a step-size parameter of the coefficient adjusting unit to a first value, when the bioimpedance signal or the potential signal satisfies the condition, and
the filter control unit controls the coefficient adjusting unit to set the step-size parameter of the coefficient adjusting unit to a second value that is smaller than the first value, when the bioimpedance signal or the potential signal does not satisfy the condition.

19. The biological information providing apparatus according to any one of claims 1 to 8, wherein
when a magnitude the bioimpedance signal or a magnitude of the potential signal exceed a predetermined first threshold value, the filter control unit outputs, as a biological information signal, a signal according to the potential signal before it is determined that the bioimpedance signal or the potential signal satisfies the condition, and when the magnitude of the bioimpedance signal or the magnitude of the potential signal does not exceed the predetermined first threshold value, the filter control unit outputs the potential signal as the biological information signal, and
the filter control unit controls the coefficient adjusting unit to set a step-size parameter of the coefficient adjusting unit to a first value when the magnitude of the bioimpedance signal or the magnitude of the potential signal is equal to or less than the first threshold value and exceeds a second threshold value that is smaller than the first threshold value, and
the filter control unit controls the coefficient adjusting unit to set the step-size parameter of the coefficient adjusting unit to a second value that is less than the first value when the magnitude of the bioimpedance signal or the magnitude of the potential signal is equal to or less than the second threshold value.

20. The biological information providing apparatus according to any one of claims 1 to 8, wherein
the filter control unit controls the coefficient adjusting unit to set a step-size parameter of the coefficient adjusting unit to a first value when a magnitude of the bioimpedance signal or a magnitude of the potential signal exceeds a predetermined first threshold value,
the filter control unit controls the coefficient adjusting unit to set the step-size parameter of the coefficient adjusting unit to a second value that is less than the first value, when the magnitude of the bioimpedance signal or the magnitude of the potential signal is equal to or less than the first threshold value and exceeds a second threshold value that is smaller than the first threshold value, and
the filter control unit controls the coefficient adjusting unit to set the step-size parameter of the coefficient adjusting unit to a third value that is smaller than the second value, when the magnitude of the bioimpedance signal or the magnitude of the potential signal is equal to or less than the second threshold value.

21. The biological information providing apparatus according to any one of claims 1 to 8, wherein
in a case where the coefficient adjusting unit uses an NLMS algorithm to perform coefficient adjustment,
the filter control unit controls the coefficient adjusting unit to set a stability constant of the coefficient adjusting unit to a first value, when the bioimpedance signal or the potential signal satisfies the condition, and
the filter control unit controls the coefficient adjusting unit to set the stability constant of the adaptive filter to a second value that is greater than the first value, when or the bioimpedance signal or the potential signal does not satisfy the condition.

22. The biological information providing apparatus according to any one of claims 1 to 8, wherein
the filter control unit controls the coefficient adjusting unit such that an operation processing speed of the adaptive filter is a first operation processing speed, when the bioimpedance signal or the potential signal satisfies the condition, and
the filter control unit controls the coefficient adjusting unit such that the operation processing speed of the adaptive filter is a second operation processing speed that is slower than the first operation processing speed, when the bioimpedance signal or the potential signal does not satisfy the condition.

23. The biological information providing apparatus according to any one of claims 1 to 8, wherein
the filter control unit controls the coefficient adjusting unit such that an update interval of the coefficient is a first interval, when the bioimpedance signal or the potential signal satisfies the condition, and
the filter control unit controls the coefficient adjusting unit such that the update interval of the coefficient is a second interval that is longer than the first interval, when the bioimpedance signal or the potential signal does not satisfy the condition.

24. A biological information providing apparatus comprising:
a potential signal measuring unit which measures a potential signal through a pair of electrodes in contact with a living body; and
a selecting unit which performs selection to output, when the potential signal satisfies a predetermined condition, a signal according to the potential signal before it is determined that the potential signal satisfies the condition, as a biological information signal, and to output the potential signal as the biological information signal, when the potential signal does not satisfy the predetermined condition.

25. The biological information providing apparatus according to claim 24, further comprising:
an envelope extraction unit which performs envelope extraction on the potential signal to output a reference waveform signal;
a potential signal determination unit which determines that the potential signal satisfies a predetermined condition when a magnitude of the potential signal exceeds a magnitude of the reference waveform signal;
a phase delaying unit which delays a phase of the potential signal input to the selecting unit by a delay time due to the envelope extraction; and
a noise removal unit which outputs a signal according to the potential signal before it is determined that the potential signal satisfies the condition, when the potential signal satisfies a predetermined condition.

26. The biological information providing apparatus according to claim 24, further comprising:
an envelope extraction unit which performs envelope extraction on the potential signal to output a reference waveform signal;
a potential signal determination unit which determines that the potential signal satisfies a predetermined condition, when a magnitude of the potential signal exceeds a magnitude of the reference waveform signal;
a first phase delaying unit which delays a phase of the potential signal input to the selecting unit by a delay time due to the envelope extraction;
a noise removal unit which, when the potential signal satisfies a predetermined condition, performs low pass filtering on an output signal of the first phase delaying unit and outputs a signal according to the potential signal before it is determined that the potential signal satisfies the condition; and
a second phase delaying unit which delays a phase of the potential signal input to the selecting unit by a delay time due to the low pass filtering.
